(19) United States
(12) Patent Application Publication
(10) Pub. No.: US 2023/0159912 A1
(43) Pub. Date: May 25, 2023

(54) COMPOSITIONS AND METHODS FOR NUCLEIC ACID NORMALIZATION

(71) Applicant: BIOO Scientific Corporation, Austin, TX (US)

(72) Inventors: William Law, Austin, TX (US); Kerry Gunning, Austin, TX (US); Aracele Martinez-Mendez, Austin, TX (US)

(21) Appl. No.: 17/993,150

(22) Filed: Nov. 23, 2022

Related U.S. Application Data
(60) Provisional application No. 63/282,469, filed on Nov. 23, 2021.

(51) Int. Cl. C12N 15/10; C12Q 1/6806

(52) U.S. Cl. CPC C12N 15/1013; C12Q 1/6806

(57) ABSTRACT

Methods, compositions, and kits of the present disclosure for normalizing a mass amount of nucleic acids in each of a plurality of test samples are provided, for use in methods of nucleic acid analysis, such that substantially equal amounts of nucleic acids are present in the samples to be analyzed.

### REFERENCE TO RELATED APPLICATION

This application claims priority to U.S. Provisional Application Ser. No. 63/282,469, filed Nov. 23, 2021, the entire content of which is incorporated herein by reference.

### FIELD OF THE INVENTION

Aspects described herein relate to methods, compositions, and kits for normalizing a mass amount of nucleic acids in each of a plurality of test samples which allow a user to avoid hands-on quantitation of each individual test sample.

### BACKGROUND OF THE INVENTION

Highly sophisticated analysis of nucleic acids, such as Next-Generation Sequencing (NGS), are powerful for a number of reasons, at least one of which is that many thousands of samples can be subject to analysis simultaneously. However, in practice, the power of such cutting edge techniques can be limited by the current dearth of similarly sophisticated methods for ensuring that substantially equal amounts of nucleic acids are present in all samples to be analyzed. Current methods require hands-on quantitation of each individual sample, requiring significant time and effort.

There is a continuing need for methods, compositions, and kits for normalizing a mass amount of nucleic acids in each of a plurality of test samples.

### SUMMARY OF THE INVENTION

Methods of normalizing a mass amount of nucleic acids in each of a plurality of test samples are provided according to aspects of the present disclosure which include: 1) providing a plurality of input samples containing nucleic acids in an aqueous liquid, each of the plurality of input samples present in a separate container; 2) adding a binding mixture to each container, producing a normalization mixture in each container, wherein the binding mixture includes: i) a quantity of magnetic particles comprising pendant hydroxyl functional groups, ii) a chelating agent, iii) a binding buffer, and iv) an alcohol, wherein the binding buffer includes a buffered aqueous solution of a chaotrope, wherein the quantity of magnetic particles is capable of reversibly and non-specifically binding nucleic acids at a binding capacity in the range of 1 about nanogram to about 5 micrograms, and wherein each of the plurality of input samples contains a mass amount of nucleic acids greater than the binding capacity of the quantity of magnetic particles; 3) incubating each normalization mixture under binding conditions, thereby reversibly and non-specifically binding a portion of the nucleic acids to the magnetic particles; 4) separating the magnetic particles with the reversibly and non-specifically bound nucleic acids from non-bound nucleic acids by application of a magnetic field; and 5) eluting the reversibly and non-specifically bound nucleic acids from the magnetic particles, producing a plurality of test samples, wherein each of the plurality of test samples comprises isolated nucleic acids having a mass amount, wherein the mass amount of the isolated nucleic acids is approximately equivalent to the binding capacity of the magnetic particles, thereby providing a normalized mass amount of nucleic acids in each of the plurality of test samples.

According to aspects of methods of normalizing a mass amount of nucleic acids in each of a plurality of test samples of the present disclosure, no internal standard is added to the container.

According to aspects of methods of normalizing a mass amount of nucleic acids in each of a plurality of test samples of the present disclosure, the mass amount of nucleic acids in the input samples is not quantitated.

According to aspects of methods of normalizing a mass amount of nucleic acids in each of a plurality of test samples of the present disclosure, the magnetic particles do not include a binding partner which specifically binds to the nucleic acids.

According to aspects of methods of normalizing a mass amount of nucleic acids in each of a plurality of test samples of the present disclosure, the magnetic particles do not include a binding partner which is a nucleic acid, biotin, avidin, an antibody, an aptamer, a receptor, or a receptor ligand.

According to aspects of methods of normalizing a mass amount of nucleic acids in each of a plurality of test samples of the present disclosure, further included is 6) pooling the plurality of test samples, producing pooled test samples, and sequencing at least some of the nucleic acids in the pooled test samples.

According to aspects of methods of normalizing a mass amount of nucleic acids in each of a plurality of test samples of the present disclosure, the chaotrope includes one or more of: urea, guanidinium bromide (guanidine hydrobromide or guanidine monohydrobromide), guanidinium iodide (guanidine hydroiodide), guanidinium chloride (guanidine hydrochloride), guanidine thiocyanate (guanidinium thiocyanate), guanidine nitrate (guanidinium nitrate), guanidine sulfate salt (guanidinium sulfate), guanidine carbonate salt (guanidinium carbonate), sodium iodide, and sodium perchlorate.

According to aspects of methods of normalizing a mass amount of nucleic acids in each of a plurality of test samples of the present disclosure, the binding buffer does not include PEG.

According to aspects of methods of normalizing a mass amount of nucleic acids in each of a plurality of test samples of the present disclosure, the magnetic particles do not comprise carboxyl functional moieties and/or amine functional moieties.

According to aspects of methods of normalizing a mass amount of nucleic acids in each of a plurality of test samples of the present disclosure, the magnetic particles having pendant hydroxyl functional groups include hydroxyl-functionalized spacers covalently bonded to, and extending from the magnetic particles and/or a coating of the magnetic particles, wherein the spacer includes a chain of at least 3 atoms covalently bonded to a hydroxyl functional group.

According to aspects of methods of normalizing a mass amount of nucleic acids in each of a plurality of test samples of the present disclosure, the chelating agent is one or more of: diethylenetriaminepentaacetic acid (DTPA), ethylenediaminetetraacetic acid (EDTA), ethylene glycol-bis(β-aminoethyl ether)-N,N,N′,N′-tetraacetic acid (EGTA) and N,N-bis(carboxymethyl)glycine (NTA).

According to aspects of methods of normalizing a mass amount of nucleic acids in each of a plurality of test samples of the present disclosure, the alcohol is one or more of: methanol, ethanol and isopropanol.

According to aspects of methods of normalizing a mass amount of nucleic acids in each of a plurality of test samples of the present disclosure, the binding buffer includes a buffered aqueous solution of guanidine hydrochloride and potassium acetate or sodium acetate, and has a pH in the range of about pH 4 to about pH 6.

According to aspects of methods of normalizing a mass amount of nucleic acids in each of a plurality of test samples of the present disclosure, further included is 7) recovering the non-bound nucleic acids of one or more normalization mixtures. According to aspects of methods of the present disclosure, recovering the non-bound nucleic acids of one or more normalization mixtures includes 7a) transferring the non-bound nucleic acids of one or more normalization mixtures to corresponding containers; 7b) adding a binding mixture to each corresponding container, 7c) producing a recovery mixture in each corresponding container, the binding mixture comprising: a quantity of magnetic particles comprising pendant hydroxyl functional groups, a chelating agent, a binding buffer, and an alcohol, wherein the binding buffer comprises a buffered aqueous solution of a chaotrope, wherein the quantity of magnetic particles is capable of reversibly and non-specifically binding nucleic acids at a binding capacity in the range of about 1 nanogram to about 5 micrograms, and wherein each of the recovery mixtures contains a mass amount of nucleic acids greater than, less than, or equal to, the binding capacity of the quantity of magnetic particles; 7d) incubating each recovery mixture under binding conditions, thereby reversibly and non-specifically binding all, or a portion of, the nucleic acids to the magnetic particles, producing magnetic particles reversibly and non-specifically bound to the recovered nucleic acids; 7e) separating the magnetic particles reversibly and non-specifically bound to the recovered nucleic acids by application of a magnetic field; and 7f) eluting the reversibly and non-specifically bound recovered nucleic acids from the magnetic particles.

According to aspects of methods of normalizing a mass amount of nucleic acids in each of a plurality of test samples of the present disclosure, eluting the nucleic acids from the magnetic particles includes incubating the beads in elution buffer.

According to aspects of methods of normalizing a mass amount of nucleic acids in each of a plurality of test samples of the present disclosure, the elution buffer includes 10 mM Tris-HCl, pH 8.0, and 0.1 mM EDTA.

According to aspects of methods of normalizing a mass amount of nucleic acids in each of a plurality of test samples of the present disclosure, further included is: washing the magnetic particles reversibly and non-specifically bound to the nucleic acids with a wash liquid after application of the magnetic field and prior to elution.

According to aspects of methods of normalizing a mass amount of nucleic acids in each of a plurality of test samples of the present disclosure, the wash liquid includes about 80% ethanol. According to aspects of methods of normalizing a mass amount of nucleic acids in each of a plurality of test samples of the present disclosure, the volume:volume ratio of the alcohol to the binding buffer is in the range of about 0.25:1 to about 1.75:1. According to aspects of methods of normalizing a mass amount of nucleic acids in each of a plurality of test samples of the present disclosure, the volume:volume ratio of the alcohol to the binding buffer is in the range of about 1:1 to about 1.25:1. According to aspects of methods of normalizing a mass amount of nucleic acids in each of a plurality of test samples of the present disclosure, the volume:volume ratio of the alcohol to the binding buffer is in the range of about 1.125:1.

Kits according to aspects of the present disclosure include: magnetic particles comprising pendant hydroxyl functional groups; a binding buffer comprising a buffered aqueous solution of a chaotrope; a wash liquid; and an elution buffer. According to aspects of the present disclosure, the magnetic particles having pendant hydroxyl functional groups include a hydroxyl-functionalized spacer covalently bonded to, and extending from the magnetic particle and/or a coating of the magnetic particle, wherein the spacer comprises a chain of at least 3 atoms covalently bonded to a hydroxyl functional group.

According to aspects of the present disclosure, the elution buffer includes 10 mM Tris-HCl, pH 8.0, and 0.1 mM EDTA. According to aspects of the present disclosure, the wash liquid includes about 60% to about 100% ethanol.

Kits according to aspects of the present disclosure include: magnetic particles comprising pendant hydroxyl functional groups; a binding buffer comprising a buffered aqueous solution of a chaotrope; a wash liquid; an elution buffer, a chelating agent and an alcohol. According to aspects of the present disclosure, the magnetic particles having pendant hydroxyl functional groups include a hydroxyl-functionalized spacer covalently bonded to, and extending from the magnetic particle and/or a coating of the magnetic particle, wherein the spacer comprises a chain of at least 3 atoms covalently bonded to a hydroxyl functional group.

According to aspects of the present disclosure, the elution buffer includes 10 mM Tris-HCl, pH 8.0, and 0.1 mM EDTA. According to aspects of the present disclosure, the wash liquid includes ethanol in a concentration of about 80% ethanol.

Kits according to aspects of the present disclosure include: magnetic particles comprising pendant hydroxyl functional groups; a binding buffer including a buffered aqueous solution of guanidine hydrochloride and potassium acetate or sodium acetate, the binding buffer having a pH in the range of about pH 4 to about pH 6; a wash liquid; and an elution buffer. According to aspects of the present disclosure, the magnetic particles having pendant hydroxyl functional groups include a hydroxyl-functionalized spacer covalently bonded to, and extending from the magnetic particle and/or a coating of the magnetic particle, wherein the spacer comprises a chain of at least 3 atoms covalently bonded to a hydroxyl functional group.

According to aspects of the present disclosure, the elution buffer includes 10 mM Tris-HCl, pH 8.0, and 0.1 mM EDTA. According to aspects of the present disclosure, the wash liquid includes ethanol in a concentration of about 80% ethanol.

Kits according to aspects of the present disclosure include: magnetic particles comprising pendant hydroxyl functional groups; a binding buffer including a buffered aqueous solution of guanidine hydrochloride and potassium acetate or sodium acetate, the binding buffer having a pH in the range of about pH 4 to about pH 6; a wash liquid; an elution buffer, a chelating agent and an alcohol. According to aspects of the present disclosure, the magnetic particles having pendant hydroxyl functional groups include a hydroxyl-functionalized spacer covalently bonded to, and extending from the magnetic particle and/or a coating of the magnetic particle, wherein the spacer comprises a chain of at least 3 atoms covalently bonded to a hydroxyl functional group.

According to aspects of the present disclosure, the elution buffer includes 10 mM Tris-HCl, pH 8.0, and 0.1 mM EDTA. According to aspects of the present disclosure, the wash liquid includes ethanol in a concentration of about 80% ethanol.

Compositions for use in a method of normalizing a mass amount of nucleic acids in each of a plurality of test samples, are provided according to aspects of the present disclosure which include: an input sample or a plurality of input samples, each input sample containing nucleic acids in an aqueous liquid, each of the plurality of input samples present in a separate container; and a binding mixture in each container, producing a normalization mixture in each container, the binding mixture comprising: a quantity of magnetic particles comprising pendant hydroxyl functional groups, a chelating agent, a binding buffer, and an alcohol, wherein the binding buffer comprises a buffered aqueous solution of a chaotrope, wherein the quantity of magnetic particles is capable of reversibly and non-specifically binding nucleic acids at a binding capacity in the range of about 1 nanogram to about 5 micrograms, and wherein each of the plurality of input samples contains a mass amount of nucleic acids greater than the binding capacity of the quantity of magnetic particles.

Compositions for use in a method of normalizing a mass amount of nucleic acids in each of a plurality of test samples, are provided according to aspects of the present disclosure wherein the magnetic particles comprising pendant hydroxyl functional groups comprise a hydroxyl-functionalized spacer covalently bonded to, and extending from the magnetic particle and/or a coating of the magnetic particle, wherein the spacer comprises a chain of at least 3 atoms covalently bonded to a hydroxyl functional group.

Compositions for use in a method of normalizing a mass amount of nucleic acids in each of a plurality of test samples, are provided according to aspects of the present disclosure wherein no internal standard is present in the container or containers.

Compositions for use in a method of normalizing a mass amount of nucleic acids in each of a plurality of test samples, are provided according to aspects of the present disclosure wherein the magnetic particles do not include a binding partner which specifically binds to the nucleic acids.

Compositions for use in a method of normalizing a mass amount of nucleic acids in each of a plurality of test samples, are provided according to aspects of the present disclosure wherein the magnetic particles do not include a binding partner which is a nucleic acid, biotin, avidin, an antibody, an aptamer, a receptor, or a receptor ligand.

Compositions for use in a method of normalizing a mass amount of nucleic acids in each of a plurality of test samples, are provided according to aspects of the present disclosure wherein the chaotrope comprises one or more of: urea, guanidinium bromide (guanidine hydrobromide or guanidine monohydrobromide), guanidinium iodide (guanidine hydroiodide), guanidinium chloride (guanidine hydrochloride), guanidine thiocyanate (guanidinium thiocyanate), guanidine nitrate (guanidinium nitrate), guanidine sulfate salt (guanidinium sulfate), guanidine carbonate salt (guanidinium carbonate), sodium iodide, and sodium perchlorate.

Compositions for use in a method of normalizing a mass amount of nucleic acids in each of a plurality of test samples, are provided according to aspects of the present disclosure wherein the binding buffer does not include PEG.

Compositions for use in a method of normalizing a mass amount of nucleic acids in each of a plurality of test samples, are provided according to aspects of the present disclosure wherein the magnetic particles do not comprise carboxyl functional moieties and/or amine functional moieties.

Compositions for use in a method of normalizing a mass amount of nucleic acids in each of a plurality of test samples, are provided according to aspects of the present disclosure wherein the chelating agent is one or more of: diethylenetriaminepentaacetic acid (DTPA), ethylenediaminetetraacetic acid (EDTA), ethylene glycol-bis(β-aminoethyl ether)-N,N,N′,N′-tetraacetic acid (EGTA) and N,N-bis(carboxymethyl)glycine (NTA).

Compositions for use in a method of normalizing a mass amount of nucleic acids in each of a plurality of test samples, are provided according to aspects of the present disclosure wherein the alcohol is one or more of: methanol, ethanol and isopropanol.

Compositions for use in a method of normalizing a mass amount of nucleic acids in each of a plurality of test samples, are provided according to aspects of the present disclosure wherein the binding buffer comprises a buffered aqueous solution of guanidine hydrochloride and potassium acetate or sodium acetate, and has a pH in the range of about pH 4 to about pH 6.

Compositions for use in a method of normalizing a mass amount of nucleic acids in each of a plurality of test samples, are provided according to aspects of the present disclosure wherein the volume:volume ratio of the alcohol to the binding buffer is in the range of about 0.25:1 to about 1.75:1.

Compositions for use in a method of normalizing a mass amount of nucleic acids in each of a plurality of test samples, are provided according to aspects of the present disclosure wherein the volume:volume ratio of the alcohol to the binding buffer is in the range of about 1:1 to about 1.25:1.

Compositions for use in a method of normalizing a mass amount of nucleic acids in each of a plurality of test samples, are provided according to aspects of the present disclosure wherein the volume:volume ratio of the alcohol to the binding buffer is about 1.125:1.

### BRIEF DESCRIPTION OF THE DRAWINGS

FIG. 1 is a schematic illustration of methods according to aspects of the present disclosure;

FIG. 2A is a graph showing that variation in the ratio of ethanol to binding buffer results in variation in the range of sizes of DNA fragments bound to, and eluted from, the magnetic particles having pendant hydroxyl functional groups; increasing ethanol results in an increase in isolation of smaller DNA fragments;

FIG. 2B is an image of gel analysis of the size of the isolated DNA, showing that a different range of sizes of DNA fragments is isolated depending on the ratio of ethanol to binding buffer;

FIG. 3 shows results of gel analysis of variation in the ratio of ethanol to binding buffer; a DNA ladder (Gene Ladder 50 bp) in the far left lane compared with samples of DNA isolated using various ratios of ethanol to binding buffer shows variation in the range of sizes of DNA fragments bound to, and eluted from, the magnetic particles having pendant hydroxyl functional groups; increasing ethanol results in an increase in isolation of smaller DNA fragments;

FIG. 4 is an image of gel analysis of the size of the isolated DNA, showing that a different range of sizes of DNA fragments is isolated depending on the ratio of ethanol to binding buffer; a 1.125 ratio of ethanol to binding buffer results in a bell-curve shaped size distribution;

FIG. 5 shows results of gel analysis of variation in the ratio of ethanol to binding buffer; a DNA ladder (Gene Ladder 50 bp) in the far left lane compared with samples of DNA isolated using various ratios of the aqueous component (Aqu) of the “normalization mixture” including DNA in an aqueous liquid to binding buffer (BB) on size distribution and shows no significant effect on the range of sizes of DNA fragments bound to, and eluted from, the magnetic particles comprising pendant hydroxyl functional groups;

FIG. 6 shows results of gel analysis of variation in pH of the normalization mixture; a DNA ladder (Gene Ladder 50 bp) in the far left lane compared with samples of DNA isolated using the indicated pHs on size distribution; increasing pH has a mild effect on the range of sizes of DNA fragments bound to, and eluted from, the magnetic particles having pendant hydroxyl functional groups, excluding fragments 150 bp and smaller;

FIG. 7 is a graph showing results according to a method of the present disclosure using magnetic particles having pendant hydroxyl functional groups with various amounts of input DNA, 25 ng, 50 ng, 100 ng, 250 ng, 350 ng, 500 ng, 625 ng, and 750 ng, were used by two separate operators;

FIG. 10 shows that when the same amount of magnetic particles having pendant hydroxyl functional groups was added to each sample containing different amounts of DNA, equivalent amounts of DNA were recovered, indicating that methods of the present disclosure do provide normalization by total DNA mass;

FIG. 8 is a graph showing library recovery ratio and percent cluster balancing and demonstrating replicability and reproducibility achieved using methods according to the present disclosure; varied estimated input DNA amounts between 500 ng-1100 ng were used; magnetic particles having pendant hydroxyl functional groups used were capable of normalization at >500 ng post PCR; 100% normalization was achieved across 88 libraries by DNA amount recovered (+/−1.35 fold difference; total average DNA recovered: ˜100 ng (˜11.5 nM); approximately 94% normalization by clustering was achieved; +/−1.38 fold difference, excluding 5 libraries that fell below % cluster threshold; +/−1.48 fold difference across 88 libraries;

FIG. 9 is an image of results of gel electrophoresis using a sample of each of 24 normalized amplified libraries;

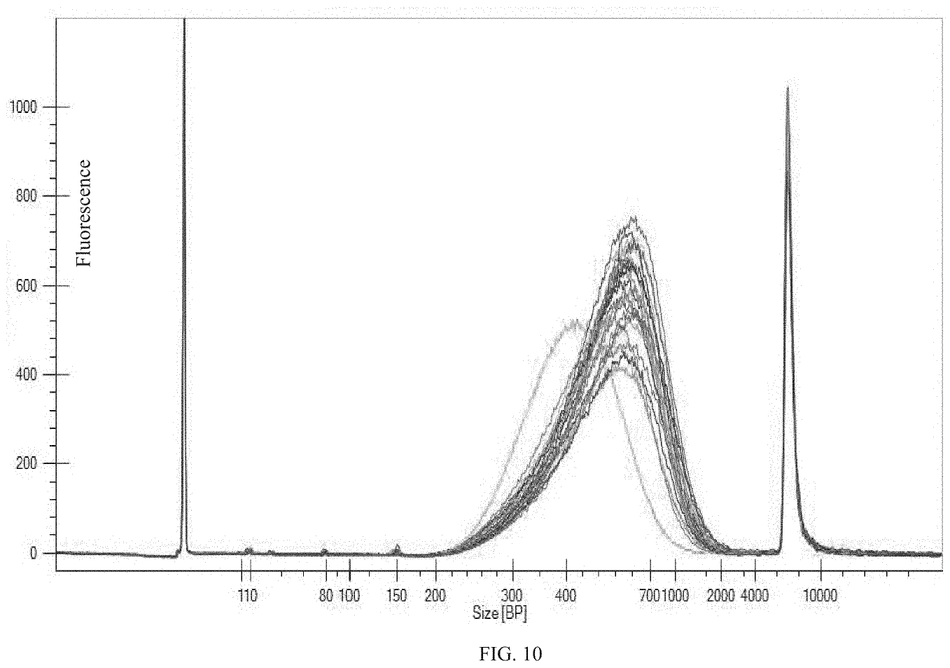

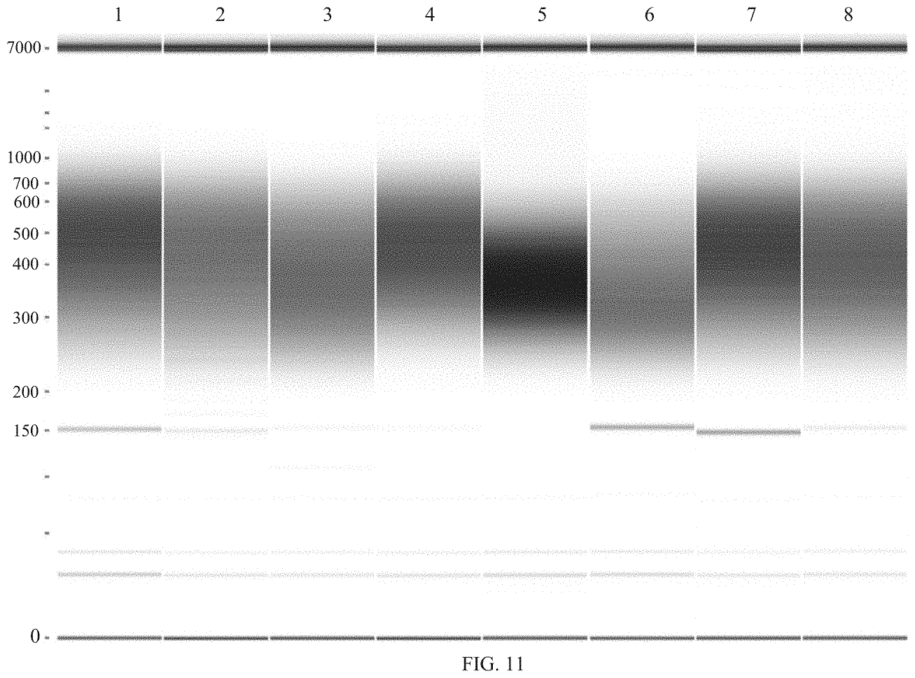

FIG. 10 is a graph of results of fragment size analysis using a sample of each of 24 normalized amplified libraries; and

FIG. 11 is an image of results of gel electrophoresis using a sample of each of 8 normalized amplified libraries.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002;Next Generation Sequencing: Methods and Protocols (Methods in Molecular Biology), 2018, Humana Press, Springer Nature; D. L. Nelson and M. M. Cox, Lehninger Principles of Biochemistry, 4th Ed., W. H. Freeman & Company, 2004; and Herdewijn, P. (Ed.), Oligonucleotide Synthesis: Methods and Applications, Methods in Molecular Biology, Humana Press, 2004.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

The term “about,” and grammatical equivalents thereof, as used herein in relation to a reference numerical value refers to the reference numerical value and numerical values within 10% of the reference numerical value, including numerical values which are plus or minus (+/−) 1%, +/−2%, +/−3%, +/−4%, +/−5%, +/−6%, +/−7%, +/−8%, +/−9%, or +/−10% of the reference numerical value. In the context of a stated range of reference numerical values, the term “about” and grammatical equivalents thereof, as used herein refers to a range which includes numerical values that are+/−1%, +/−2%, +/−3%, +/−4%, +/−5%, +/−6%, +/−7%, +/−8%, +/−9%, or +/−10% of the reference lower limit of the range of numerical values and numerical values that are+/−1%, +/−2%, +/−3%, +/−4%, +/−5%, +/−6%, +/−7%, +/−8%, +/−9%, or +/−10% of the reference upper limit of the range of numerical values. Further, unless otherwise specified, a listing of numerical values herein is understood to include all intermediate and fractional values of the listed numerical values, e.g., 50%, 60%, 75%, is understood to include 55%, 64.5%, and 74%.

Methods of normalizing a mass amount of nucleic acids in each of a plurality of test samples are provided according to aspects of the present disclosure which include: 1) providing a plurality of input samples containing nucleic acids in an aqueous liquid, each of the plurality of input samples present in a separate container; 2) adding a binding mixture to each container, producing a normalization mixture in each container, wherein the binding mixture includes: i) a quantity of magnetic particles comprising pendant hydroxyl functional groups, ii) a chelating agent, iii) a binding buffer, and iv) an alcohol, wherein the binding buffer includes a buffered aqueous solution of a chaotrope, wherein the quantity of magnetic particles is capable of reversibly and non-specifically binding nucleic acids at a binding capacity in the range of about 1 nanogram to about 5 micrograms, and wherein each of the plurality of input samples contains a mass amount of nucleic acids greater than the binding capacity of the quantity of magnetic particles; 3) incubating each normalization mixture under binding conditions, thereby reversibly and non-specifically binding a portion of the nucleic acids to the magnetic particles; 4) separating the magnetic particles with the reversibly and non-specifically bound nucleic acids from non-bound nucleic acids by application of a magnetic field; and 5) eluting the reversibly and non-specifically bound nucleic acids from the magnetic particles, producing a plurality of test samples, wherein each of the plurality of test samples comprises isolated nucleic acids having a mass amount, wherein the mass amount of the isolated nucleic acids is approximately equivalent to the binding capacity of the magnetic particles, thereby providing a normalized mass amount of nucleic acids in each of the plurality of test samples.

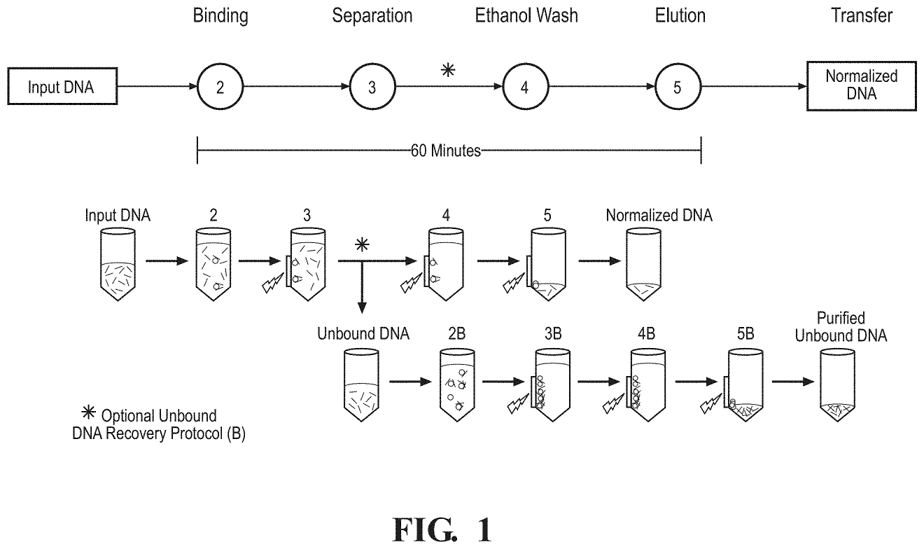

FIG. 1 illustrates methods of normalizing a mass amount of nucleic acids in each of a plurality of test samples according to aspects of the present disclosure which include: providing a plurality of input samples containing nucleic acids in an aqueous liquid, each of the plurality of input samples present in a separate container, referred to in FIG. 1 as “Input DNA.”

At 2 in FIG. 1 is diagrammatically shown binding of nucleic acids to magnetic particles comprising pendant hydroxyl functional groups following adding a binding mixture to each container, producing a normalization mixture in each container, and incubating each normalization mixture under binding conditions, thereby reversibly and non-specifically binding a portion of the nucleic acids to the magnetic particles. At 3 in FIG. 1 is diagrammatically shown separating the magnetic particles with the reversibly and non-specifically bound nucleic acids from non-bound nucleic acids by application of a magnetic field. Also shown, at the *, is removal of the supernatant containing the non-bound nucleic acids, shown here as “unbound DNA. Once the supernatant containing the non-bound nucleic acids is removed, the nucleic acids reversibly and non-specifically bound to the magnetic particles are washed with a wash solution, in this case, an ethanol wash solution as illustrated at 4.

At 5 in FIG. 1 is shown a step of eluting the reversibly and non-specifically bound nucleic acids from the magnetic particles, producing a plurality of test samples, wherein each of the plurality of test samples comprises isolated nucleic acids having a mass amount, wherein the mass amount of the isolated nucleic acids is approximately equivalent to the binding capacity of the magnetic particles, thereby providing a normalized mass amount of nucleic acids in each of the plurality of test samples, i.e. “normalized DNA” in this figure. FIG. 1 further shows optional recovery of the non-bound nucleic acids, including at 2B binding of the previously non-bound nucleic acids to magnetic particles comprising pendant hydroxyl functional groups following adding a binding mixture to each container and incubating under binding conditions, thereby reversibly and non-specifically binding a portion, or all, of the previously non-bound nucleic acids to the magnetic particles. At 3B is shown separating the magnetic particles with the reversibly and non-specifically bound nucleic acids from the supernatant by application of a magnetic field. At 4B is shown a wash step, and at 5B is shown elution of the nucleic acids, producing purified unbound nucleic acids. The term “normalizing” as used herein refers to a process which includes producing a plurality of test samples wherein each individual test sample of the plurality includes a substantially equal amount of nucleic acids compared to each other individual test sample of the plurality. The plurality of test samples is derived from a corresponding plurality of input samples containing nucleic acids. Nucleic acids in the input samples may or may not be quantitated prior to producing the corresponding plurality of test samples. Nucleic acids in the test samples may or may not be quantitated. An advantage of methods of normalizing a mass amount of nucleic acids in each of a plurality of test samples according to aspects of the present disclosure is that quantitation of the nucleic acids in the test samples is not required.

The term “substantially equal” as used herein in reference to amounts of nucleic acids in the test samples refers to amounts which are no more than 40% different, such as no more than 40%, 35%, 30%, 25%, 20%, 15%, 10%, 9%, 8%, 7%, 6%, 5% 4%, 3%, 2%, 1%, or less, different.

The term “nucleic acid” as used herein refers to a polymer of nucleotides, such as ribonucleotides and/or deoxyribonucleotides, and/or nucleotide analogs, covalently bonded to each other. Nucleic acids include, but are not limited to, DNA, such as genomic DNA, cDNA, DNA amplification produce, and/or RNA, such as mRNA, rRNA, tRNA, siRNA, miRNA, piRNA, and small RNA.

A “nucleic acid” may include standard nucleotides, typically referred to as adenine (A), guanine (G), cytidine (C), thymidine (T), and uridine (U) in reference to the base included. As used herein, the term “nucleotide” refers to a molecule that contains a base moiety, a sugar moiety, and a phosphate moiety. A nucleic acid may include one or more non-standard nucleotides, i.e. nucleotide analogs. The term “nucleotide analog” refers to a nucleotide which contains one or more modifications to the base moiety and/or the sugar moiety, and/or the phosphate moiety which modifies at least one aspect of the chemical properties of the nucleotide analog compared to a reference standard nucleotide, while retaining other properties which allow the nucleotide analog to perform its intended function. Nucleotide analogs, and nucleic acids including them, are well known in the art and may be synthesized according to standard procedures and/or obtained commercially.

According to aspects of the present disclosure, the nucleic acids in the input sample are derived from a biological sample obtained from any organism, any cell or cells derived from an organism, including a single-cell organism, a multicellular organism, a prokaryotic organism, a eukaryotic organism, an invertebrate organism, a vertebrate organism or any nucleic acid-containing entity, such as a virus or mycoplasma. According to aspects of the present disclosure, the nucleic acids in the input sample are derived from a biological sample obtained from a plant, bacterium, archaeon, or fungus. According to aspects of the present disclosure, the nucleic acids in the input sample are derived from a mammalian subject or non-mammalian subject. A biological sample obtained from a subject can be, but is not limited to, a sample of saliva, blood, plasma, serum, mucous, urine, feces, nasal material, cerebrospinal fluid, cerebroventricular fluid, pleural fluids, pulmonary and bronchial lavage samples, sweat, tears, semen, bladder wash samples, amniotic fluid, lymph, hair, skin, tumor, and peritoneal fluid.

A mammalian subject can be any mammal including, but not limited to, a human; a non-human primate; a rodent such as a mouse, rat, or guinea pig; a domesticated pet such as a cat or dog; a horse, cow, pig, sheep, goat, camel, vicuna, or rabbit, marine mammal, such as a whale, dolphin, seal, or sea lion.

A non-mammalian subject can be any non-mammal including, but not limited to, a bird, reptile, amphibian, insect, fish, or nematode.

Subjects can be either gender and can be any age. In aspects of methods of the present disclosure, the subject is human.

According to aspects of the present disclosure, the nucleic acids in the input sample are derived from an environmental sample. An environmental sample can be, but is not limited to, a liquid, gas, or solid sample, including, but not limited to, a water sample, a sewage sample, an air sample, a surface swab, a food sample, a beverage sample, a clothing sample, and a soil sample.

According to particular aspects, the input samples and test samples contain amplified DNA, such as DNA obtained by an amplification reaction. According to particular aspects, the input samples and test samples contain amplified DNA produced by polymerase chain reaction (PCR). According to particular aspects, the input samples and test samples contain amplified DNA produced by isothermal amplification. The template nucleic acids for the amplification reaction can be nucleic acids of any origin, including a biological sample or environmental sample.

Amplification of a nucleic acid is achieved using an in vitro amplification method. The term “amplification method” refers to a method for copying a template target nucleic acid, thereby producing nucleic acids which include copies of all or a portion of the template target nucleic acid.

Amplification methods included in embodiments of the present invention are those which include template directed primer extension catalyzed by a nucleic acid polymerase using a pair of primers which flank the target nucleic acid, illustratively including, but not limited to, Polymerase Chain Reaction (PCR), reverse-transcription PCR (RT-PCR). ligation-mediated PCR (LM-PCR), phi-29 PCR, real-time quantitative PCR (qPCR), whole genome amplification, and other nucleic acid amplification methods, for instance, as described in C. W. Dieffenbach et al., PCR Primer: A Laboratory Manual, Cold Spring Harbor Laboratory Press, 2003; V. Demidov et al., DNA Amplification: Current Technologies and Applications, Taylor & Francis, 2004; and Kroneis, T. (Ed.), Whole Genome Amplification: Methods and Protocols (Methods in Molecular Biology), 2015, Humana Press ISBN-10: 1493929895.

The term “isothermal amplification” refers to nucleic acid amplification that includes single temperature amplification and therefore avoids the need for thermal cycling (as in PCR). Examples of isothermal amplification include, but are not limited to, circular helicase-dependent amplification (cHDA), genome exponential amplification reaction (GEAR), helicase-dependent amplification (HDA), isothermal multiple displacement amplification (IMDA), loop-mediated isothermal amplification (LAMP), multiple displacement amplification (MDA), nicking enzyme amplification reaction (NEAR), nucleic acid sequence-based amplification (NASBA), ramification amplification (RAM), recombinase polymerase amplification (RPA), rolling circle amplification (RCA), self-sustained sequence replication (3SR), signal mediated amplification of RNA technology (SMART), strand-displacement amplification (SDA), single primer isothermal amplification (SPIA), and transcription mediated amplification (TMA).

The terms “amplified nucleic acid” and “amplified DNA” as well as plurals thereof refer to the product of a process of copying a target nucleic acid template.

Amplified nucleic acids optionally contain additional materials such as, but not limited to, nucleic acid sequences, functional groups for chemical reaction and detectable labels, present in the primers and not present in the original nucleic acid template. Such primer-derived materials add functionality such as primer binding sites for additional amplification reactions and/or a functional group for chemical bonding to a substrate.

The term “mass amount” as used herein refers to an amount of DNA expressed in units of mass, e.g., micrograms or nanograms.

Any type of container can be used in methods according to the present disclosure.

According to particular aspects, the container can be a tube, vial, chamber, well, or depression. According to particular aspects, the container can be a multi-compartment container which can hold multiple individual samples separately, each sample in an individual separate compartment, such as, but not limited to, a multi-depression slide, chip, or tray, or a multi-well plate.

According to aspects of methods of normalizing a mass amount of nucleic acids in each of a plurality of test samples of the present disclosure, no internal standard is used. According to aspects of methods of normalizing a mass amount of nucleic acids in each of a plurality of test samples of the present disclosure, no exogenous internal standard is added to, or included in, the containers in which the input samples or test samples are present.

According to aspects of methods of normalizing a mass amount of nucleic acids in each of a plurality of test samples of the present disclosure, the mass amount of nucleic acids in the input samples is not quantitated. Thus, for example, nucleic acids in the input samples are not quantitated and/or nucleic acids in the test samples are not quantitated, thereby eliminating time and labor intensive steps in production of test samples.

A plurality of test samples having normalized mass amounts of nucleic acids in each of the plurality of test samples is useful in various nucleic acid analysis procedures such as, but not limited to, sequencing, including high-throughput sequencing, methylation analysis, single-strand confirmation polymorphism, mass spectrometry, capillary electrophoresis, high resolution melt analysis, and restriction fragment length polymorphism.

A plurality of test samples having normalized mass amounts of nucleic acids in each of the plurality of test samples is useful in various nucleic acid analysis procedures such as, but not limited to, massively parallel sequencing (also called Next Generation Sequencing (NGS), polony sequencing, ion semiconductor sequencing, pyrosequencing, single-molecule real-time sequencing, sequencing by synthesis, sequencing by ligation, combinatorial probe anchor synthesis, nanopore sequencing, and chain termination sequencing.

Magnetic particles capable of reversibly and non-specifically binding nucleic acids are included in methods, compositions, and kits according to aspects of the present disclosure.

As disclosed herein, magnetic particles having pendant hydroxyl functional groups are included in methods, compositions, and kits according to aspects of the present disclosure which are capable of reversibly and non-specifically binding nucleic acids. The term “non-specific binding” as used herein with respect to magnetic particles having pendant hydroxyl functional groups refers to association of the magnetic particles with the nucleic acids due to a non-specific interaction, such as, in a non-limiting example, ionic interaction and/or hydrogen-bonding, in a binding mixture under binding conditions as described herein. The term “non-specific binding” as used herein further refers to association of the magnetic particles with the nucleic acids to a sufficient extent that the nucleic acids remain non-specifically bound to the magnetic particles when separated from non-bound nucleic acids by application of a magnetic field, removal of the supernatant, and washing with a wash liquid but the association is readily reversed by eluting the nucleic acids from the magnetic particles.

The term “magnetic” as used herein includes magnetic materials, including paramagnetic, superparamagnetic, ferromagnetic, and ferrimagnetic materials. Thus, magnetic particles capable of reversibly and non-specifically binding nucleic acids may include one or more magnetic materials, including paramagnetic, superparamagnetic, ferromagnetic, and ferrimagnetic materials. Examples of such magnetic materials include Fe, Co, CrO₂, Dy, EuO, Gd, Ni, MnAs, MnBi, NiO/Fe, NiFe₂O₄, Fe₃O₄.

The magnetic particles can be any of various shapes, regular, irregular, or a mixture of regular and irregular, such as, but not limited to, sphere, spheroidal, ellipse, ellipsoidal, rod, and rod-like. The magnetic particles can be beads.

The magnetic particles typically have an average diameter or average longest dimension size of less than 50 microns. The magnetic particles may be nanoparticles, microparticles, or a mixture thereof, wherein microparticles have an average diameter or average longest dimension size in the range of about 1 nm to about 1000 nm, and microparticles have an average diameter or average longest dimension size in the range of about 1 micron to about 50 microns. According to aspects of the present disclosure, the magnetic particles have an average diameter or average longest dimension size in the range of about 1 nm to about 1000 nm, such as about 1 nm to about 10 nm, such as about 1 nm to about 100 nm, such as about 10 nm to about 50 nm, such as about 10 nm to about 100 nm, such as about 50 nm to about 200 nm, such as about 100 nm to about 250 nm, such as about 200 nm to about 500 nm, such as about 300 nm to about 600 nm, such as about 500 nm to about 750 nm, such as about 700 nm to about 1000 nm, such as such as about 1 micron to about 10 microns, such as about 10 micron to about 20 microns, or such as about 25 micron to about 50 microns. According to aspects of the present disclosure, the magnetic particles have an average diameter or average longest dimension size in the range of about 0.5 micron to about 5 microns. According to aspects of the present disclosure, the magnetic particles have an average diameter or average longest dimension size in the range of about 1 micron to about 3 microns.

According to aspects of methods of normalizing a mass amount of nucleic acids in each of a plurality of test samples of the present disclosure, the magnetic particles include a surface which is functionalized to include hydroxyl functional groups pending from the surface, i.e., pendant hydroxyl functional groups. The magnetic particles may include a solid magnetic core encapsulated in one or more non-magnetic layers which include hydroxyl functional groups or which can be functionalized to include hydroxyl functional groups.

According to aspects of the present disclosure, the magnetic particles may include a core of fragments of magnetic material encapsulated in a polymeric material, having a surface functionalized to include hydroxyl functional groups. According to aspects of the present disclosure, the magnetic particles may include a core of fragments of magnetic material encapsulated in polyvinyl alcohol (PVA), the surface of which particles includes hydroxyl functional groups, such as by silanization.

According to aspects of the present disclosure, the magnetic particles include silica.

According to aspects of the present disclosure, the magnetic particles are silica-coated magnetic particles.

According to aspects of the present disclosure, a hydroxyl-functionalized spacer is covalently bonded to, and extends from, the magnetic particle surface and/or a coating of the magnetic particle surface, such as a silica or polymer coating such that the hydroxyl functional group is spaced away from the magnetic particle surface and/or a coating of the magnetic particle surface. According to aspects of the present disclosure, the spacer can be a multi-atom group, or a chain of atoms. A non-limiting example of a spacer which is a multi-atom group is C(O).

According to aspects of the present disclosure, a spacer is, or includes, a chain of atoms such as, branched or linear chain of 3-20, or more, atoms. According to aspects of the present disclosure, a spacer is, or includes, a linear chain of 4-20, or more, atoms. According to aspects of the present disclosure, a spacer is, or includes, a linear chain of 3, 4, 5, 6, 7, 8, 9, 10, 11, 12, 13, 14, 15, 16, 17, 18, 19, or 20 atoms. According to aspects of the present disclosure, a spacer is, or includes, a linear chain of 6, 7, 8, 9, 10, 11, or 12 atoms. According to aspects of the present disclosure, a spacer is, or includes, a linear chain of 8, 9, 10, 11, 12, 13, 14, 15, 16, 17, 18, 19, or 20 atoms. According to aspects of the present disclosure, a spacer is, or includes, a linear chain of 10, 11, 12, 13, 14, 15, 16, 17, 18, 19, or 20 atoms. According to aspects of the present disclosure, a spacer is, or includes, a linear chain of 10, 11, 12, 13, 14, or 15 atoms.

According to aspects of the present disclosure, a spacer is, or includes, a chain of atoms such as, but not limited to, substituted or unsubstituted C₃-C₂₀ alkyl, substituted or unsubstituted C₃-C₂₀ alkenyl, substituted or unsubstituted C₃-C₂₀ alkynyl, substituted or unsubstituted C₆-C₁₂ aryl, substituted or unsubstituted C₃-C₁₂ cycloalkyl, substituted or unsubstituted C₅-C₁₂ heteroaryl, or substituted or unsubstituted C₅-C₁₂ heterocyclyl, wherein the chain includes at least one hydroxyl functional group, and preferably at least one terminal hydroxyl functional group. The term “terminal hydroxyl functional group” as used herein refers to a hydroxyl group on the final atom of a chain of atoms in a spacer, i.e. the atom of the chain of atoms which is furthest from the magnetic particle, or within no more than 2, 3, or 4 atoms from the final atom of the chain of atoms in a spacer.

According to aspects of the present disclosure, a spacer is, or includes, a chain of atoms such as, but not limited to, substituted or unsubstituted C₃ alkyl, substituted or unsubstituted C₄ alkyl, substituted or unsubstituted C₅ alkyl, substituted or unsubstituted C₆ alkyl, substituted or unsubstituted C₇ alkyl, substituted or unsubstituted C₅ alkyl, substituted or unsubstituted C₉ alkyl, substituted or unsubstituted C₁₀ alkyl, substituted or unsubstituted C₁₁ alkyl, substituted or unsubstituted C₁₂ alkyl, substituted or unsubstituted C₁₃ alkyl, substituted or unsubstituted C₁₄ alkyl, substituted or unsubstituted C₁₅ alkyl, substituted or unsubstituted C₁₆ alkyl, substituted or unsubstituted C₁₇ alkyl, substituted or unsubstituted C₁₅ alkyl, substituted or unsubstituted C₁₉ alkyl, or substituted or unsubstituted C₂₀ alkyl, wherein the chain includes at least one hydroxyl functional group, and preferably at least one terminal hydroxyl functional group. Magnetic particles including hydroxyl functional groups pending from the surface, i.e., pendant hydroxyl functional groups, can be made by well-known techniques, or purchased commercially, see e.g. U.S. Pat. No. 7,129,308 and Yang, H., et al., ACS Appl. Mater. Interfaces, 2015, 7, 1, 774-781.

According to aspects of methods of normalizing a mass amount of nucleic acids in each of a plurality of test samples of the present disclosure, a quantity of magnetic particles capable of reversibly and non-specifically binding nucleic acids at a binding capacity in the range of about 1 nanogram to about 5 micrograms is included in the binding mixture, wherein each of the plurality of input samples contains a mass amount of nucleic acids greater than the binding capacity of the quantity of magnetic particles. Binding capacity of the quantity of magnetic particles can be determined using any of various methods, including, but not limited to, empirical determination, and/or estimation and/or prediction based on characteristics of the magnetic particles. Thus, for example, binding capacity of the magnetic particles can be empirically determined using known amounts of the magnetic particles with known amounts of nucleic acids to determine the amount of nucleic acids which binds to the magnetic particles under defined binding conditions. In a further example, binding capacity can be estimated or predicted by calculation, using characteristics of the magnetic particles including surface area of the magnetic particles, density of the functional groups on the magnetic particles, and the number of functional groups associated with an individual nucleic acid molecule non-specifically bound to a magnetic particle. Thus, for example, assuming a surface area of 3.14×10⁶ nm for a 1 micron diameter magnetic particle, assuming a density of 1 hydroxyl functional groups pendant from the surface of the magnetic particle per n^{m} of surface area of the magnetic particle, with an estimate of 1 to 10 hydroxyl functional groups pendant from the surface of the magnetic particle involved in the non-specific binding of a nucleic acid molecule, the binding capacity of a single magnetic particle would be predicted to be in the range from approximately 30,000 to 300,000 nucleic acid molecules per 1 micron diameter magnetic particle. Binding capacity using different magnetic particle sizes, different functional group density and different nucleic acid molecule size can be similarly estimated.

According to aspects of methods of normalizing a mass amount of nucleic acids in each of a plurality of test samples of the present disclosure, the magnetic particles do not include pendant carboxyl functional moieties and/or pendant amine functional moieties.

According to aspects of methods of normalizing a mass amount of nucleic acids in each of a plurality of test samples of the present disclosure, the magnetic particles do not include a binding partner which specifically binds to the nucleic acids. According to aspects, the term “binding partner which specifically binds to the nucleic acids” refers to a protein, peptide, and/or nucleic acid capable of specific binding to the nucleic acids. For example, the magnetic particles do not include a binding partner which is a nucleic acid complementary to a nucleic acid in the input sample or test sample, avidin, an antibody, an aptamer, a receptor, or a receptor ligand. According to aspects of methods of normalizing a mass amount of nucleic acids in each of a plurality of test samples of the present disclosure, the magnetic particles do not include biotin.

According to aspects of methods of normalizing a mass amount of nucleic acids in each of a plurality of test samples of the present disclosure, a step of adding a binding mixture to each container, producing a normalization mixture in each container is included.

The binding mixture includes: i) a quantity of magnetic particles having pendant hydroxyl functional groups, ii) a chelating agent, iii) a binding buffer, and iv) an alcohol.

The quantity of magnetic particles having pendant hydroxyl functional groups included in the binding mixture is sufficient to reversibly and non-specifically bind nucleic acids at a binding capacity in the range of about 1 nanogram to about 5 micrograms. The quantity of magnetic particles having pendant hydroxyl functional groups included in the binding mixture can be calculated, for example by determining the binding capacity of the magnetic particles having pendant hydroxyl functional groups and taking into account the amount of nucleic acid desired to be in the test samples

A chelating agent included in the binding mixture according to aspects of the present disclosure is present in the binding mixture in a concentration of about 0.5 mM to about 5 mM. A chelating agent included in the binding mixture according to aspects of the present disclosure is one or more of: diethylenetriaminepentaacetic acid (DTPA), ethylenediaminetetraacetic acid (EDTA), ethylene glycol-bis(β-aminoethyl ether)-N,N,N′,N′-tetraacetic acid (EGTA) and N,N-bis(carboxymethyl)glycine (NTA).

The binding buffer included in the binding mixture includes a buffered aqueous solution of a chaotrope. An included chaotrope may be one or more of: urea, guanidinium bromide (guanidine hydrobromide or guanidine monohydrobromide), guanidinium iodide (guanidine hydroiodide), guanidinium chloride (guanidine hydrochloride), guanidine thiocyanate (guanidinium thiocyanate), guanidine nitrate (guanidinium nitrate), guanidine sulfate salt (guanidinium sulfate), guanidine carbonate salt (guanidinium carbonate), sodium iodide, and sodium perchlorate. A chaotrope included in the binding buffer according to aspects of the present disclosure is present in the binding buffer in a concentration of about 0.1 M to about 6 M.

The buffer included in the binding buffer may be an acetate buffer, including potassium acetate or sodium acetate, adjusted with acetic acid to have a pH in the range of about pH 4 to about pH 6.

According to aspects of the present disclosure, the binding buffer does not include PEG.

According to aspects of the present disclosure, the binding buffer includes a buffered aqueous solution of guanidine hydrochloride and potassium acetate or sodium acetate, and has a pH in the range of about pH 4 to about pH 6.

An alcohol included in the binding mixture includes one or more of: methanol, ethanol and isopropanol.

According to aspects of the present disclosure, the volume:volume ratio of the alcohol to the binding buffer in the binding mixture is in the range of about 0.25:1 to about 1.75:1. According to aspects of methods of normalizing a mass amount of nucleic acids in each of a plurality of test samples of the present disclosure, the volume:volume ratio of the alcohol to the binding buffer is in the range of about 1:1 to about 1.25:1. According to aspects of methods of normalizing a mass amount of nucleic acids in each of a plurality of test samples of the present disclosure, the volume:volume ratio of the alcohol to the binding buffer is about 1.125:1.

According to aspects of the present disclosure, the average size of the nucleic acids bound to the magnetic particles can be controlled by varying the volume:volume ratio of the alcohol to the binding buffer.

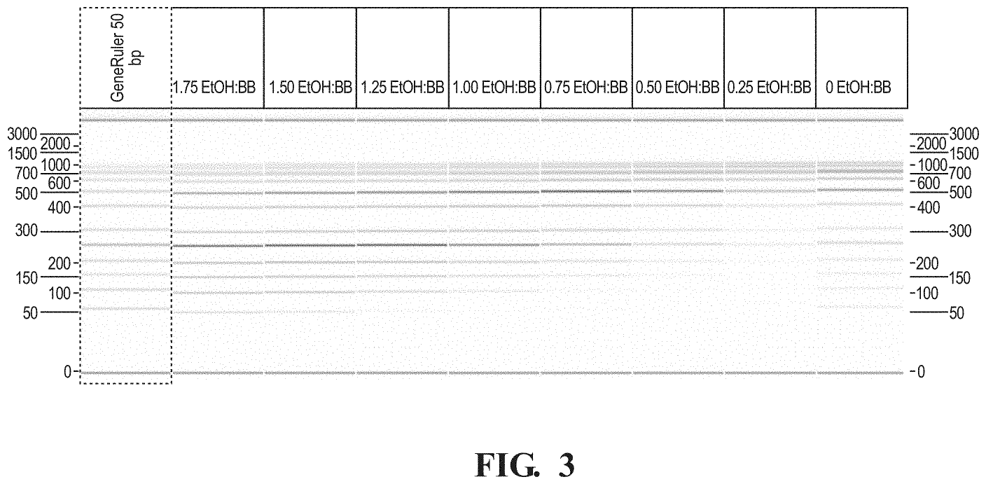

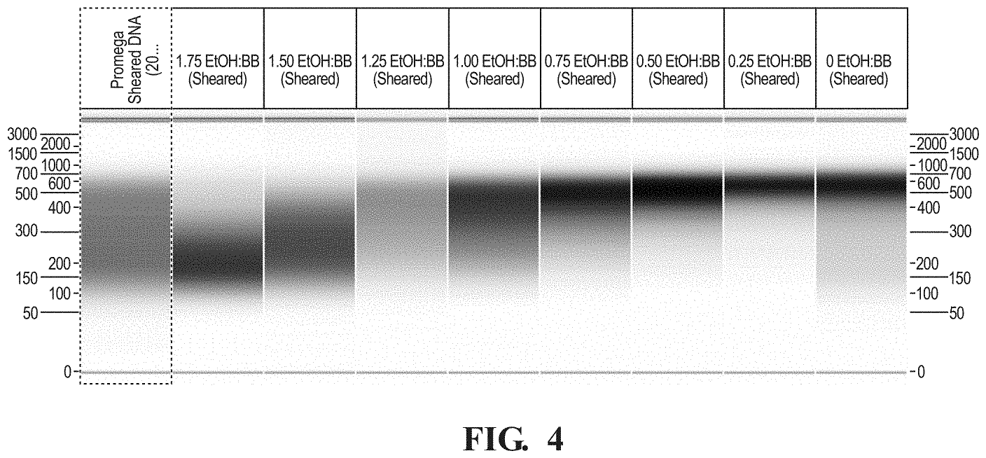

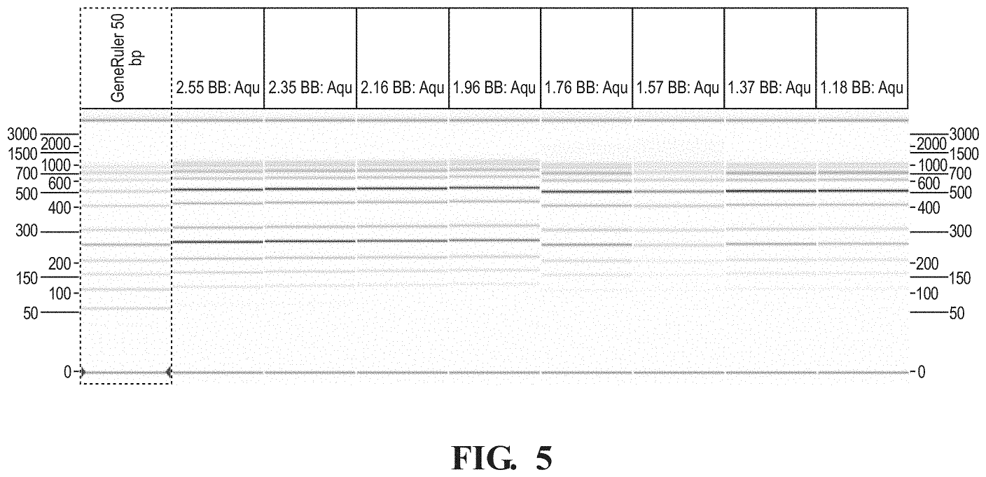

As illustrated in FIGS. 3 and 4, a higher volume:volume ratio of the alcohol to the binding buffer selects for binding of smaller nucleic acids (enriched for an average size of about 100 bp to about 300 bp in length for 1.75:1 alcohol:binding buffer) to the magnetic beads and therefore selective recovery of the smaller nucleic acids in the final test samples. Conversely, a lower volume:volume ratio of the alcohol to the binding buffer selects for binding of larger nucleic acids (about 500 bp to about 1000 bp in length for 0.25:1 alcohol:binding buffer) to the magnetic beads and therefore selective recovery of the larger nucleic acids in the final test samples. Therefore, appropriate selection of volume:volume ratio of the alcohol to the binding buffer in the binding mixture in the range of 0.25:1 to 1.75:1 can be made to selectively recover nucleic acids of a desired size.

As noted, adding a binding mixture to each container produces a normalization mixture in each container. The normalization mixture in each container is then incubated under binding conditions, promoting binding of nucleic acids in the normalization mixture to the magnetic particles. Binding conditions include a temperature in the range of about 15° C. to about 30° C., for a time in the range of about 5 minutes to about 2 hours, such as at a temperature in the range of about 20° C. to about 25° C. for a time in the range of about 5 minutes to about 15 minutes. The temperature may be higher or lower, such as in the range of about 4° C. to about 40° C., and the time of incubation may be accordingly adjusted to be longer or shorter.

Separating the magnetic particles with the reversibly and non-specifically bound nucleic acids from non-bound nucleic acids in each container may be accomplished by application of a magnetic field. The supernatant can then be removed, leaving the magnetic particles with the reversibly and non-specifically bound nucleic acids in each container, i.e. separated from the non-bound nucleic acids.

Once the magnetic particles with the reversibly and non-specifically bound nucleic acids are separated from the non-bound nucleic acids, the reversibly and non-specifically bound nucleic acids are eluted from the magnetic particles, producing a plurality of test samples. According to aspects of the present disclosure, eluting the nucleic acids from the magnetic particles includes incubating the beads in elution buffer. According to aspects of the present disclosure an “elution buffer” is an aqueous liquid that promotes dissociation of the non-specifically bound nucleic acids from the magnetic particles such that the nucleic acids are released into the elution buffer, without significant degradation of the nucleic acids. The elution buffer can be an aqueous liquid, such as, but not limited to, 10 mM Tris-HCl, pH 7.0-8.5, 10 mM Tris-Acetate, pH 7.0-8.5, saline, phosphate buffered saline, water, and may further include EDTA such as 0.1 mM EDTA According to aspects of the present disclosure, the elution buffer includes 10 mM Tris-HCl, pH 8.0, and 0.1 mM EDTA.

Following elution, each of the plurality of test samples generated thereby includes isolated nucleic acids having a mass amount, wherein the mass amount of the isolated nucleic acids is substantially equivalent to the binding capacity of the magnetic particles, thereby providing a normalized mass amount of nucleic acids in each of the plurality of test samples.

According to aspects of methods of normalizing a mass amount of nucleic acids in each of a plurality of test samples of the present disclosure, further included is: washing the magnetic particles reversibly and non-specifically bound to the nucleic acids with a wash liquid after application of the magnetic field and prior to elution. According to aspects of methods of normalizing a mass amount of nucleic acids in each of a plurality of test samples of the present disclosure, the wash liquid includes ethanol in the range of about 60% to about 100%. According to aspects of methods of normalizing a mass amount of nucleic acids in each of a plurality of test samples of the present disclosure, the wash liquid includes an aqueous solution of ethanol containing about 60% ethanol, about 70% ethanol, about 80% ethanol, about 90% ethanol, or more. According to aspects of methods of normalizing a mass amount of nucleic acids in each of a plurality of test samples of the present disclosure, the wash liquid includes an aqueous solution of about 80% ethanol.

According to aspects of the present disclosure, once the test samples are obtained, two or more of the test samples can be pooled prior to analysis. According to aspects of the present disclosure, once the test samples are obtained, two or more of the test samples can be pooled equally or unequally by volume, as desired, prior to analysis. One or more methods of nucleic acid analysis can be performed on the pooled samples.

According to aspects of the present disclosure, the non-bound nucleic acids of one or more normalization mixtures can be recovered for later use. The recovered non-bound nucleic acids of one or more normalization mixtures can be stored and/or used in any of various nucleic acid analyses. According to aspects of methods of the present disclosure, recovering the non-bound nucleic acids of one or more normalization mixtures includes transferring the non-bound nucleic acids of one or more normalization mixtures to corresponding containers; adding a binding mixture to each corresponding container, producing a recovery mixture in each corresponding container, the binding mixture comprising: a quantity of magnetic particles having pendant hydroxyl functional groups, a chelating agent, a binding buffer, and an alcohol, wherein the binding buffer comprises a buffered aqueous solution of a chaotrope, wherein the quantity of magnetic particles is capable of reversibly and non-specifically binding nucleic acids at a binding capacity in the range of 1 nanogram to 5 micrograms, and wherein each of the recovery mixtures contains a mass amount of nucleic acids greater than, less than, or equal to, the binding capacity of the quantity of magnetic particles; incubating each recovery mixture under binding conditions, thereby reversibly and non-specifically binding all, or a portion of, the nucleic acids to the magnetic particles, producing magnetic particles reversibly and non-specifically bound to the recovered nucleic acids; separating the magnetic particles reversibly and non-specifically bound to the recovered nucleic acids by application of a magnetic field; and eluting the reversibly and non-specifically bound recovered nucleic acids from the magnetic particles.

Kits according to aspects of the present disclosure include: magnetic particles having pendant hydroxyl functional groups; a binding buffer including a buffered aqueous solution of a chaotrope; a wash liquid; and an elution buffer. According to aspects of the present disclosure, the elution buffer includes 10 mM Tris-HCl, pH 8.0, and 0.1 mM EDTA. According to aspects of the present disclosure, the wash liquid includes about 80% ethanol.

Kits according to aspects of the present disclosure include: magnetic particles having pendant hydroxyl functional groups; a binding buffer including a buffered aqueous solution of a chaotrope; a wash liquid; an elution buffer, a chelating agent and an alcohol. According to aspects of the present disclosure, the elution buffer includes 10 mM Tris-HCl, pH 8.0, and 0.1 mM EDTA. According to aspects of the present disclosure, the wash liquid includes about 80% ethanol.

Kits according to aspects of the present disclosure include: magnetic particles having pendant hydroxyl functional groups; a binding buffer including a buffered aqueous solution of guanidine hydrochloride and potassium acetate or sodium acetate, the binding buffer having a pH in the range of about pH 4 to about pH 6; a wash liquid; and an elution buffer. According to aspects of the present disclosure, the elution buffer includes 10 mM Tris-HCl, pH 8.0, and 0.1 mM EDTA. According to aspects of the present disclosure, the wash liquid includes about 80% ethanol.

Kits according to aspects of the present disclosure include: magnetic particles having pendant hydroxyl functional groups; a binding buffer including a buffered aqueous solution of guanidine hydrochloride and potassium acetate or sodium acetate, the binding buffer having a pH in the range of about pH 4 to about pH 6; a wash liquid; an elution buffer, a chelating agent and an alcohol. According to aspects of the present disclosure, the elution buffer includes 10 mM Tris-HCl, pH 8.0, and 0.1 mM EDTA. According to aspects of the present disclosure, the wash liquid includes about 60% to about 100% ethanol. According to aspects of the present disclosure, the wash liquid includes an aqueous solution of ethanol wherein the ethanol is about 60%, about 70%, about 80%, about 90%, about 95%, or more, in the aqueous solution. According to aspects of the present disclosure, the wash liquid is an aqueous solution including about 80% ethanol.

Embodiments of inventive compositions and methods are illustrated in the following examples. These examples are provided for illustrative purposes and are not considered limitations on the scope of inventive compositions and methods.

### EXAMPLES

### Materials and Methods Used in the Examples with Variations where Indicated

To make 36 mL of pH adjusted potassium acetate:

TABLE 1
pH Adjusted Potassium Acetate (pH ~5.2)

| | |
|---|---|
| 5M Potassium Acetate | 24 mL |
| Glacial Acetic Acid | 12 mL |

Transfer ˜10 mL of the solution to a clean tube, and determine the pH. pH is between 5.1-5.3

To make 50 mL of binding buffer:

TABLE 2

| Binding Buffer | | | | | | |
|---|---|---|---|---|---|---|
| Components | Total | | Pipette Gun | | Manual Pipette | |
| 6M Guanidine | 36.25 | mL | 36 | mL | 250 | μL |
| Hydrochloride | | | | | | |
| pH Adjusted | 8.71 | mL | 8 | mL | 710 | μL |
| Potassium Acetate | | | | | | |
| (pH ~5.2) | | | | | | |
| Ultrapure Water | 5.04 | mL | 5 | mL | 40 | μL |

Final concentrations of binding buffer components in this example: 4.35 M guanidine hydrochloride, 0.58 M potassium acetate, acetic acid was used to adjust the final pH to pH 4.1-4.4.

Transfer ˜10 mL of the solution to a clean tube, and determine the pH. pH is between 4.15-4.35

Wash Buffer

80% ethanol

Elution Buffer

10 mM Tris-HCl, pH 8.0, 0.1 mM EDTA.

Master binding mixture for each reaction container containing 50 μL sample including nucleic acids to be normalized:
25 μL 20 mM EDTA
1 μL magnetic particles comprising pendant hydroxyl functional groups (22.5 μg/μL)-22.5 μg total
80 μL Binding Buffer
90 μL 100% Ethanol

### Example 1

Make a master binding mixture containing:
25 μL 20 mM EDTA
1 μL magnetic particles comprising pendant hydroxyl functional groups (22.5 μg/μL)-22.5 μg total
80 μL Binding Buffer
90 μL 100% Ethanol for each reaction container containing 50 μL PCR reaction to be normalized.

Once the master binding mixture is prepared, vortex the tube containing it thoroughly (>5 seconds). Ensure the beads are equally distributed.

Add 196 μL (single channel) of the master binding mixture to each reaction container (tube) containing 50 microliters of an aqueous DNA-containing solution (input sample) and pipette=3 cycles (cycle=aspirate/dispense).

Once the master binding mixture has been aliquoted to each reaction container, forming a normalization mixture, set a multichannel pipette to 150 μL and pipette the mixture 10 cycles to fully distribute the beads in the normalization mixture.

Additional mixing may be necessary to properly distribute the beads.

Incubate the normalization mixture undisturbed for 10 minutes at room temperature.

After 10 minutes of incubation, place the tube on a magnet for 1 minute. The time may vary depending on strength of magnet. Leave beads on magnet until the solution is clear

Remove supernatant with a multichannel pipette set to 200 μL—Note: Total volume of supernatant is ˜246 μL. Two aspirations is required to remove the total supernatant.

If recovery of unbound DNA is desired, transfer the supernatant to a clean tube. Add 3 μL of 50 mg/mL magnetic particles comprising pendant hydroxyl functional groups, mix=10 cycles with a pipette to fully disperse the beads, and then incubate for 10 minutes undisturbed at room temperature.

If unbound DNA recovery is unnecessary, discard supernatant.

Once the solution in the tubes is clear, indicating separation of the magnetic particles comprising pendant hydroxyl functional groups with the bound nucleic acids from non-bound nucleic acids by application of a magnetic field, keep the tubes in contact with the magnet and, add 200 μL of freshly made 80% ethanol to wash the magnetic particles comprising pendant hydroxyl functional groups with the bound nucleic acids.

Incubate on the magnet at room temperature for 30 seconds.

Remove and discard the supernatant.

Repeat 80% ethanol wash step by adding 200 μL 80% ethanol, incubate on magnet at room temperature for 30 seconds, and remove and discard supernatant. Using a clean set of p20 multichannel pipette tips, set the p20 volume to 15 μL and carefully remove any residual ethanol from the bottom of the tubes.

Leave the tubes on the magnet for 3 minutes to air dry the pellet which contains the magnetic particles comprising pendant hydroxyl functional groups with the bound nucleic acids. Leave caps of tubes open.

Remove tubes from the magnet and add 22 μL of Elution Buffer to the pellet and pipette repeatedly until all beads are fully resuspended (10 pipette cycles).

Incubate off the magnet at room temperature for 2 minutes.

Place tube on magnet for 2 minutes. The time may vary depending on strength of magnet. Leave beads on magnet until the solution is clear.

Transfer 20 μL of the supernatant to a new tube.

Expected DNA recovery ˜100-150ng total (˜5-6.5ng/μL)

If sequencing, pool equal volumes of all libraries, great than or equal to 2 μL minimum per library.

Mix the combined library thoroughly. Quantify final pool by Qubit. Assess final pool size by LabChip.

### Example 2

Effect of various ratios of ethanol to binding buffer on size distribution of DNA captured

The method of Example 1 was followed with substitution of various ratios of ethanol to binding buffer. An equivalent amount, 400 ng, of sheared DNA was included in each input sample.

The master binding mixture used contained:
25 μL 20 mM EDTA
1 μL magnetic particles comprising pendant hydroxyl functional groups (22.5 μg/μL)-22.5 μg total
80 μL Binding Buffer
10 μL, 20 μL, 30 μL, or 40 μL 100% Ethanol, for each reaction container containing the sample of 400 ng sheared DNA in 50 μL aqueous liquid.

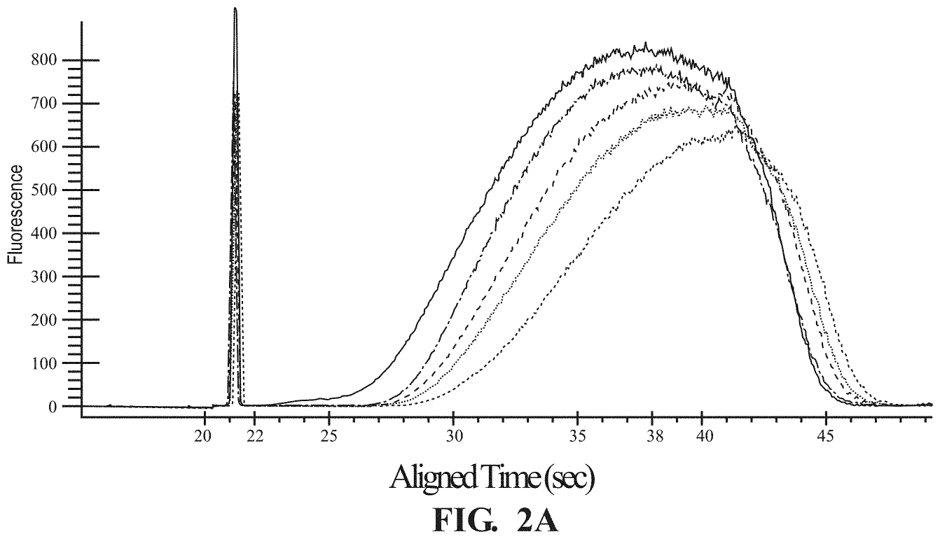

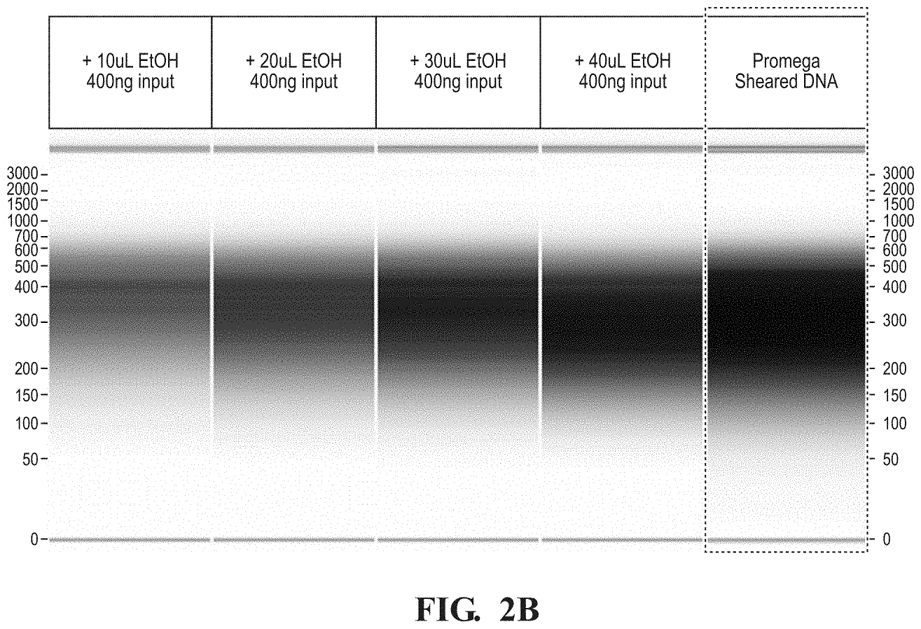

As shown in the graph in FIG. 2A, variation in the ratio of ethanol to binding buffer results in variation in the range of sizes of DNA fragments bound to, and eluted from, the magnetic particles comprising pendant hydroxyl functional groups. Increasing ethanol results in an increase in isolation of smaller DNA fragments. FIG. 2B is an image of gel analysis of the size of the isolated DNA, showing that a different range of sizes of DNA fragments is isolated depending on the ratio of ethanol to binding buffer.

### Example 3

Effect of various ratios of ethanol to binding buffer on size distribution of DNA captured

The method of Example 1 was followed with substitution of various ratios of ethanol (EtOH) to binding buffer (BB). An equivalent amount, 400 ng, of sheared DNA was included in each input sample.

The master binding mixture used contained:
25 μL 20 mM EDTA
1 μL magnetic particles comprising pendant hydroxyl functional groups (22.5 μg/μL)-22.5 μg total
80 μL Binding Buffer (BB)
100% Ethanol in a volume to achieve a ratio of 0.25:1 ethanol:binding buffer, 0.50: ethanol:binding buffer, 0.75:1 ethanol:binding buffer, 1.00:1 ethanol:binding buffer, 1.25:1 ethanol:binding buffer, 1.50:1 ethanol:binding buffer, 1.75:1 ethanol:binding buffer for each reaction container containing the sample of 400 ng sheared DNA in 50 μL aqueous liquid. Ethanol was omitted as a control.

FIG. 3 shows results of gel analysis of variation in the ratio of ethanol to binding buffer. A DNA ladder (Gene Ladder 50 bp) in the far left lane compared with samples of DNA isolated using various ratios of ethanol to binding buffer shows variation in the range of sizes of DNA fragments bound to, and eluted from, the magnetic particles comprising pendant hydroxyl functional groups. Increasing ethanol results in an increase in isolation of smaller DNA fragments. FIG. 4 is an image of gel analysis of the size of the isolated DNA, showing that a different range of sizes of DNA fragments is isolated depending on the ratio of ethanol to binding buffer. A 1.125 ratio of ethanol to binding buffer results in a bell-curve shaped size distribution. Table 3 shows the average size in base pairs (bp) of sheared DNA fragments isolated using various ratios of ethanol to binding buffer.

TABLE 3

| Ethanol:Binding | Sheared DNA |
|---|---|
| Buffer Ratio | Average bp Size |
| 1.75 EtOH:BB | 265 |
| 1.50 EtOH:BB | 297 |
| 1.25 EtOH:BB | 378 |
| 1.00 EtOH:BB | 375 |
| 0.75 EtOH:BB | 423 |
| 0.50 EtOH:BB | 473 |
| 0.25 EtOH:BB | 518 |
| 0 EtOH:BB | 439 |

### Example 4

Effect of the aqueous component (Aqu) of the “normalization mixture” including DNA in an aqueous liquid to binding buffer (BB) on size distribution of DNA captured.

The method of Example 1 was followed with substitution of various ratios of binding buffer to the aqueous component. An equivalent amount, 400 ng, of sheared DNA was included in each input sample.

The master binding mixture used contained:
25 μL 20 mM EDTA
1 μL magnetic particles comprising pendant hydroxyl functional groups (22.5 μg/μL)-22.5 μg total
Binding Buffer in a volume to achieve a ratio of 2.55:1 binding buffer:Aqu, 2.35: binding buffer:Aqu, 2.16:1 binding buffer:Aqu, 1.96:1 binding buffer:Aqu, 1.76:1 binding buffer:Aqu, 1.57:1 binding buffer:Aqu, 1.37:1 binding buffer:Aqu, 1.18:1 binding buffer:Aqu.
90 μL 100% Ethanol for each reaction container containing the sample of 400 ng sheared DNA in 50 μL aqueous liquid.

FIG. 5 shows results of gel analysis of variation in the ratio of ethanol to binding buffer. A DNA ladder (Gene Ladder 50 bp) in the far left lane compared with samples of DNA isolated using various ratios of the aqueous component (Aqu) of the “normalization mixture” including DNA in an aqueous liquid to binding buffer (BB) on size distribution and shows no significant effect on the range of sizes of DNA fragments bound to, and eluted from, the magnetic particles comprising pendant hydroxyl functional groups.

### Example 5

Effect of pH of the normalization mixture on size distribution of DNA captured.

The method of Example 1 was followed with variation in pH. An equivalent amount, 400 ng, of sheared DNA was included in each input sample.

The master binding mixture used contained:
25 μL 20 mM EDTA
1 μL magnetic particles comprising pendant hydroxyl functional groups (22.5 μg/μL)-22.5 μg total
80 μL Binding Buffer
90 μL 100% Ethanol for each reaction container containing the sample of 400 ng sheared DNA in 50 μL aqueous liquid. The final normalization mixture had a pH between 4.15-4.35 (“standard”), pH 5.0, pH 5.5, or pH 6.0.

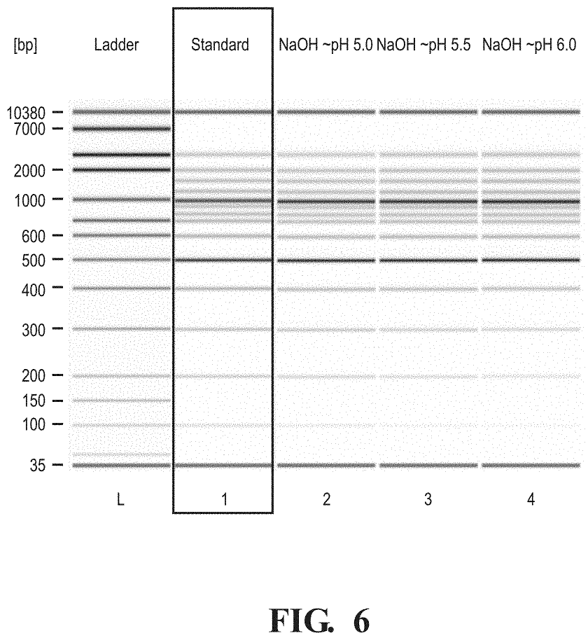

FIG. 6 shows results of gel analysis of variation in pH of the normalization mixture. A DNA ladder (Gene Ladder 50 bp) in the far left lane compared with samples of DNA isolated using the indicated pHs on size distribution. Increasing pH has a mild effect on the range of sizes of DNA fragments bound to, and eluted from, the magnetic particles comprising pendant hydroxyl functional groups, excluding fragments 150 bp and smaller.

### Example 6

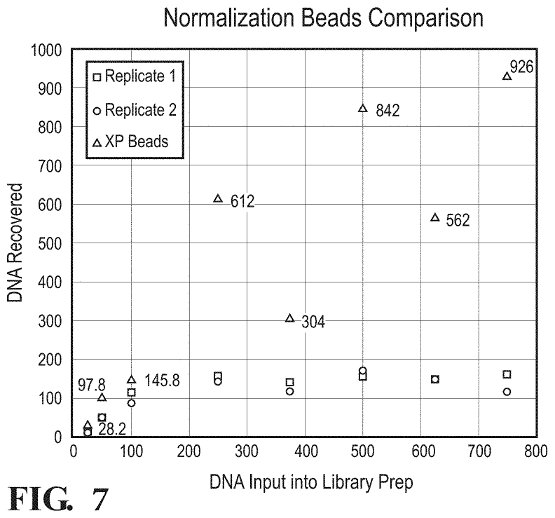

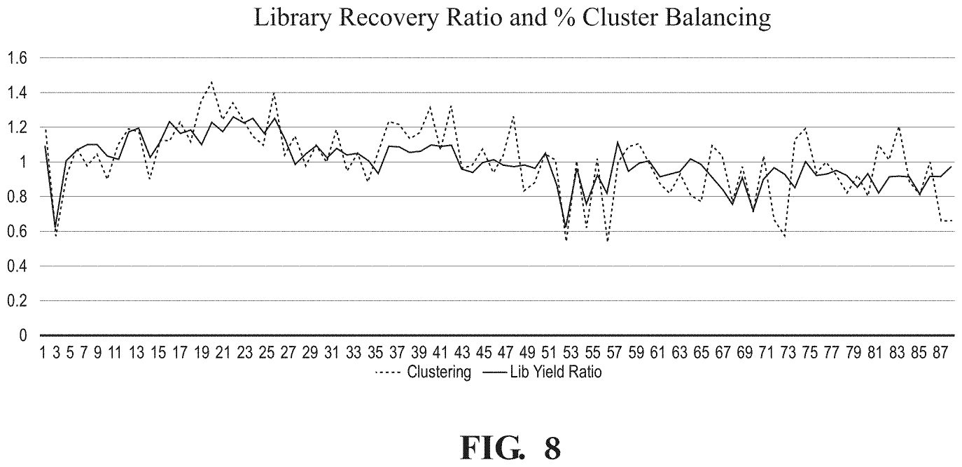

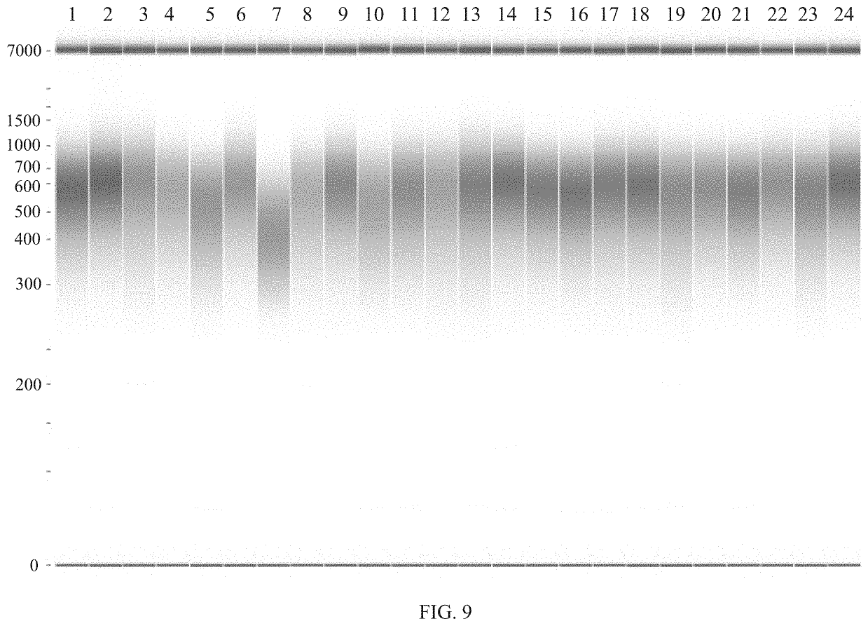

A method of the present disclosure was used with various amounts of input DNA, 25 ng, 50 ng, 100 ng, 250 ng, 350 ng, 500 ng, 625 ng, and 750 ng, were used by two separate operators. However, as can be seen in FIG. 7, when adding the same amount of magnetic particles comprising pendant hydroxyl functional groups to each sample containing different amounts of DNA, equivalent amounts of DNA were recovered, indicating that this product and procedure do provide normalization by total DNA mass.

XP beads are carboxylate beads in a PEG:NaCl solution, designed to bind all DNA in the tube, washed to remove salts, and eluted. They are the standard bead and buffer type used to purify DNA, a commercially available example of which is Beckman Agencourt Beads.

FIG. 8 is a graph showing library recovery ratio and percent cluster balancing and demonstrating replicability and reproducibility achieved using methods according to the present disclosure. Varied estimated input DNA amounts between 500 ng-1100 ng were used. Magnetic particles comprising pendant hydroxyl functional groups used were capable of normalization at >500 ng post PCR. 100% normalization was achieved across 88 libraries by DNA amount recovered (+/−1.35 fold difference. Total average DNA recovered: ˜100 ng (˜11.5 nM) Approximately 94% normalization by clustering was achieved; +/−1.38 fold difference, excluding 5 libraries that fell below % cluster threshold; +/−1.48 fold difference across 88 libraries.

### Example 7

Bead-Based Library Normalization

In this example, 24 samples of genomic DNA were used to generate 24 Amplified Libraries. For this, 10 ng genomic DNA input material was used and fragmentation time was 10 minutes. Amplification included 7 cycles of PCR, generating the 24 amplified libraries. These were then normalized as follows:

Magnetic normalization beads comprising pendant hydroxyl functional groups were suspended in 100% ethanol producing a magnetic bead concentration of 22.5 μg/μL.

The magnetic normalization beads were then mixed with binding buffer by adding 1 μL (22.5 μg/μL, i.e. 22.5 μg total) magnetic particles comprising pendant hydroxyl functional groups to 108 μL binding buffer. The binding buffer used in this example contained 3.31M Guanidine HCl, 0.44M potassium acetate, and 4.76M EDTA.

An ethanol:binding buffer master mixture containing magnetic beads was generated by mixing 75 uL of 100% ethanol with 109 μL binding buffer/magnetic beads. The volume:volume ratio of 100% ethanol:binding buffer was 0.69:1.

For normalization, 25 μL of each amplified library was deposited in a separate well of a 96-well PCR plate and then 184 μL of master mixture containing magnetic normalization beads was added to each well. The final concentration of EDTA was 2.79 mM. The materials in each well were then thoroughly mixed until homogenized followed by incubation at room temperature for 8 minutes.

The 96-well PCR plate was then placed on a magnetic stand at room temperature for 5 minutes until the supernatant appeared completely clear and the magnetic normalization beads were pelleted at the bottom of the wells.

The clear supernatant was then removed without disturbing the pellet of magnetic normalization beads. Once removed, the supernatant, containing non-normalized library, was transferred to a clean tube and frozen. The tube may be thawed and purified for additional recovery, if desired.

With the 96-well plate containing the pellets of magnetic normalization beads still on the magnetic stand, 200 μL of 80% ethanol was added to each magnetic normalization bead pellet and the plate was then incubated at room temperature for 30 seconds. The ethanol supernatant was then carefully removed by pipette. This was repeated for a total of two washes with 80% ethanol. Once the ethanol was removed, the washed pellet of magnetic normalization beads was air dried at room temperature for 3 minutes.

The dried magnetic normalization beads were then resuspended with 23 μL of elution buffer in each well and mixed thoroughly until homogenized. The elution buffer used in this example was a solution of 10 mM Tris-HCl, pH 8.0 and 0.1 mM EDTA. The resuspended magnetic normalization beads were then incubated at room temperature for 2 minutes.

Following incubation, the 96-well PCR plate was placed on the magnetic stand at room temperature for 2 minutes after which the supernatant appeared completely clear.

20 μL of the clear supernatant (eluted sample) was transferred from each well to a corresponding well of a new 96-well plate.

5 μL of each eluted sample was removed and pooled with the others in a single 1.5 mL tube producing a Pooled Library. Quantification of the pooled library can be performed using fluorometric methods to determine concentration. qPCR may be used to quantify DNA library template for optimal cluster density.

A sample of the pooled library was examined by electrophoresis to ensure the desired library sizing was obtained. A fold difference of 1.20 across the 24 Amplified Libraries following normalization, see FIG. 9 and FIG. 10. FIG. 9 is an image of results of gel electrophoresis using a sample of each of the 24 normalized Amplified Libraries. FIG. 10 is a graph of results of fragment size analysis using a sample of each of the 24 normalized Amplified Libraries.

At this stage, the pooled library was used for cluster generation per the standard Illumina® protocol.

### Example 8

Bead-Based Library Normalization

In this example, 8 samples of genomic DNA were used to generate 8 Amplified Libraries. For this, 10 ng genomic DNA input material was used and fragmentation time was 10 minutes. Amplification included 7 cycles of PCR, generating the 8 amplified libraries. These were then normalized as follows:

Magnetic normalization beads comprising pendant hydroxyl functional groups were suspended in 100% ethanol producing a magnetic bead concentration of 22.5 μg/μL.

The magnetic normalization beads were then mixed with binding buffer by adding 1 μL (22.5 μg/μL, i.e. 22.5 μg total) magnetic particles comprising pendant hydroxyl functional groups to 108 μL binding buffer. The binding buffer used in this example contained 3.31M Guanidine HCl, 0.44M potassium acetate, and 4.76M EDTA.

An ethanol:binding buffer master mixture containing magnetic beads was generated by mixing 75 uL of 100% ethanol with 109 μL binding buffer/magnetic beads. The volume:volume ratio of 100% ethanol:binding buffer was 0.69:1.

For normalization, 25 μL of each amplified library was deposited in a separate well of a 96-well PCR plate and then 184 μL of master mixture containing magnetic normalization beads was added to each well. The final concentration of EDTA was 2.79 mM. The materials in each well were then thoroughly mixed until homogenized followed by incubation at room temperature for 8 minutes.

The 96-well PCR plate was then placed on a magnetic stand at room temperature for 5 minutes until the supernatant appeared completely clear and the magnetic normalization beads were pelleted at the bottom of the wells.

The clear supernatant was then removed without disturbing the pellet of magnetic normalization beads. Once removed, the supernatant, containing non-normalized library, was transferred to a clean tube and frozen. The tube may be thawed and purified for additional recovery, if desired.

With the 96-well plate containing the pellets of magnetic normalization beads still on the magnetic stand, 200 μL of 80% ethanol was added to each magnetic normalization bead pellet and the plate was then incubated at room temperature for 30 seconds. The ethanol supernatant was then carefully removed by pipette. This was repeated for a total of two washes with 80% ethanol. Once the ethanol was removed, the washed pellet of magnetic normalization beads was air dried at room temperature for 3 minutes.

The dried magnetic normalization beads were then resuspended with 23 μL of elution buffer in each well and mixed thoroughly until homogenized. The elution buffer used in this example was a solution of 10 mM Tris-HCl, pH 8.0 and 0.1 mM EDTA. The resuspended magnetic normalization beads were then incubated at room temperature for 2 minutes.

Following incubation, the 96-well PCR plate was placed on the magnetic stand at room temperature for 2 minutes after which the supernatant appeared completely clear.

20 μL of the clear supernatant (eluted sample) was transferred from each well to a corresponding well of a new 96-well plate.

5 μL of each eluted sample was removed and pooled with the others in a single 1.5 mL tube producing a Pooled Library. Quantification of the pooled library can be performed using fluorometric methods to determine concentration. qPCR may be used to quantify DNA library template for optimal cluster density.

A sample of the pooled library was examined by electrophoresis to ensure the desired library sizing was obtained. A fold difference of 1.38 across the 8 Amplified Libraries following normalization, see FIG. 11. FIG. 11 is an image of results of gel electrophoresis using a sample of each of the 8 normalized Amplified Libraries.

At this stage, the pooled library was used for cluster generation per the standard Illumina® protocol.

Items

Item 1. A method of normalizing a mass amount of nucleic acids in each of a plurality of test samples, comprising: providing a plurality of input samples containing nucleic acids in an aqueous liquid, each of the plurality of input samples present in a separate container; adding a binding mixture to each container, producing a normalization mixture in each container, the binding mixture comprising: a quantity of magnetic particles comprising pendant hydroxyl functional groups, a chelating agent, a binding buffer, and an alcohol, wherein the binding buffer comprises a buffered aqueous solution of a chaotrope, wherein the quantity of magnetic particles is capable of reversibly and non-specifically binding nucleic acids at a binding capacity in the range of about 1 nanogram to about 5 micrograms, and wherein each of the plurality of input samples contains a mass amount of nucleic acids greater than the binding capacity of the quantity of magnetic particles; incubating each normalization mixture under binding conditions, thereby reversibly and non-specifically binding a portion of the nucleic acids to the magnetic particles; separating the magnetic particles with the reversibly and non-specifically bound nucleic acids from non-bound nucleic acids by application of a magnetic field; and eluting the reversibly and non-specifically bound nucleic acids from the magnetic particles, producing a plurality of test samples, wherein each of the plurality of test samples comprises isolated nucleic acids having a mass amount, wherein the mass amount of the isolated nucleic acids is approximately equivalent to the binding capacity of the magnetic particles, thereby providing a normalized mass amount of nucleic acids in each of the plurality of test samples.

Item 2. The method of item 1, wherein no internal standard is added to the container.

Item 3. The method of item 1 or item 2, wherein the mass amount of nucleic acids in the input samples is not quantitated.

Item 4. The method of any of items 1 to 3, wherein the magnetic particles do not include a binding partner which specifically binds to the nucleic acids.

Item 5. The method of any of items 1 to 4, wherein the magnetic particles do not include a binding partner which is a nucleic acid, biotin, avidin, an antibody, an aptamer, a receptor, or a receptor ligand.

Item 6. The method of any of items 1 to 5, further comprising pooling the plurality of test samples, producing pooled test samples, and sequencing at least some of the nucleic acids in the pooled test samples.

Item 7. The method of any of items 1 to 6, wherein the chaotrope comprises one or more of: urea, guanidinium bromide (guanidine hydrobromide or guanidine monohydrobromide), guanidinium iodide (guanidine hydroiodide), guanidinium chloride (guanidine hydrochloride), guanidine thiocyanate (guanidinium thiocyanate), guanidine nitrate (guanidinium nitrate), guanidine sulfate salt (guanidinium sulfate), guanidine carbonate salt (guanidinium carbonate), sodium iodide, and sodium perchlorate.

Item 8. The method of any of items 1 to 7, wherein the binding buffer does not include PEG.

Item 9. The method of any of items 1 to 8, wherein the magnetic particles do not comprise carboxyl functional moieties and/or amine functional moieties.

Item 10. The method of any of items 1 to 9, wherein the chelating agent is one or more of: diethylenetriaminepentaacetic acid (DTPA), ethylenediaminetetraacetic acid (EDTA), ethylene glycol-bis(β-aminoethyl ether)-N,N,N′,N′-tetraacetic acid (EGTA) and N,N-bis(carboxymethyl)glycine (NTA).

Item 11. The method of any of items 1 to 10, wherein the alcohol is one or more of: methanol, ethanol and isopropanol.

Item 12. The method of any of items 1 to 11, wherein the binding buffer comprises a buffered aqueous solution of guanidine hydrochloride and potassium acetate or sodium acetate, and has a pH in the range of about pH 4 to about pH 6.

Item 13. The method of any of items 1 to 12, further comprising: recovering the non-bound nucleic acids of one or more normalization mixtures.

Item 14. The method of any of items 1 to 13, further comprising transferring the non-bound nucleic acids of one or more normalization mixtures to corresponding containers; adding a binding mixture to each corresponding container, producing a recovery mixture in each corresponding container, the binding mixture comprising: a quantity of magnetic particles comprising pendant hydroxyl functional groups, a chelating agent, a binding buffer, and an alcohol, wherein the binding buffer comprises a buffered aqueous solution of a chaotrope, wherein the quantity of magnetic particles is capable of reversibly and non-specifically binding nucleic acids at a binding capacity in the range of about 1 nanogram to about 5 micrograms, and wherein each of the recovery mixtures contains a mass amount of nucleic acids greater than, less than, or equal to, the binding capacity of the quantity of magnetic particles; incubating each recovery mixture under binding conditions, thereby reversibly and non-specifically binding all, or a portion of, the nucleic acids to the magnetic particles, producing magnetic particles reversibly and non-specifically bound to the recovered nucleic acids; separating the magnetic particles reversibly and non-specifically bound to the recovered nucleic acids by application of a magnetic field; and eluting the reversibly and non-specifically bound recovered nucleic acids from the magnetic particles.

Item 15. The method of any of items 1 to 14, wherein eluting the nucleic acids from the magnetic particles comprises incubating the beads in elution buffer.

Item 16. The method of any of items 1 to 15, wherein the elution buffer comprises 10 mM Tris-HCl, pH 8.0, and 0.1 mM EDTA.

Item 17. The method of any of items 1 to 16, further comprising washing the magnetic particles reversibly and non-specifically bound to the nucleic acids with a wash liquid after application of the magnetic field and prior to elution.

Item 18. The method of item 17, wherein the wash liquid comprises about 60% to about 100% ethanol.

Item 19. The method of any of items 1 to 18, wherein the volume:volume ratio of the alcohol to the binding buffer is in the range of about 0.25:1 to about 1.75:1.

Item 20. The method of any of items 1 to 19, wherein the volume:volume ratio of the alcohol to the binding buffer is in the range of about 1:1 to about 1.25:1.

Item 21. The method of any of items 1 to 20, wherein the volume:volume ratio of the alcohol to the binding buffer is about 1.125:1.

Item 22. The method of any of items 1 to 21, wherein the magnetic particles comprising pendant hydroxyl functional groups comprise a hydroxyl-functionalized spacer covalently bonded to, and extending from the magnetic particle and/or a coating of the magnetic particles, wherein the spacer comprises a chain of at least 3 atoms covalently bonded to a hydroxyl functional group.

Item 23. A kit, comprising: magnetic particles comprising pendant hydroxyl functional groups; a binding buffer comprising a buffered aqueous solution of a chaotrope; a wash liquid; and an elution buffer.

Item 24. The kit of item 23, further comprising a chelating agent and an alcohol.

Item 25. The kit of item 23 or 24, wherein the binding buffer comprises a buffered aqueous solution of guanidine hydrochloride and potassium acetate or sodium acetate, and has a pH in the range of about pH 4 to about pH 6.

Item 26. The kit of any of items 23 to 25, wherein the elution buffer comprises 10 mM Tris-HCl, pH 8.0, and 0.1 mM EDTA.

Item 27. The kit of any of items 23 to 26, wherein the wash liquid comprises about 60% to about 100% ethanol.

Item 28. The kit of any of items 23 to 27, wherein the magnetic particles comprising pendant hydroxyl functional groups comprise a hydroxyl-functionalized spacer covalently bonded to, and extending from the magnetic particle and/or a coating of the magnetic particle, wherein the spacer comprises a chain of at least 3 atoms covalently bonded to a hydroxyl functional group.

Item 29. Compositions for use in a method of normalizing a mass amount of nucleic acids in each of a plurality of test samples, comprising: an input sample or a plurality of input samples, each input sample containing nucleic acids in an aqueous liquid, each of the plurality of input samples present in a separate container; and a binding mixture in each container, producing a normalization mixture in each container, the binding mixture comprising: a quantity of magnetic particles comprising pendant hydroxyl functional groups, a chelating agent, a binding buffer, and an alcohol, wherein the binding buffer comprises a buffered aqueous solution of a chaotrope, wherein the quantity of magnetic particles is capable of reversibly and non-specifically binding nucleic acids at a binding capacity in the range of about 1 nanogram to about 5 micrograms, and wherein each of the plurality of input samples contains a mass amount of nucleic acids greater than the binding capacity of the quantity of magnetic particles.

Item 30. The composition of item 29, wherein the magnetic particles comprising pendant hydroxyl functional groups comprise a hydroxyl-functionalized spacer covalently bonded to, and extending from the magnetic particle and/or a coating of the magnetic particle, wherein the spacer comprises a chain of at least 3 atoms covalently bonded to a hydroxyl functional group.

Item 31. The composition of item 29 or item 30, wherein no internal standard is present in the container or containers.

Item 32. The composition of any of items 29 to 31, wherein the magnetic particles do not include a binding partner which specifically binds to the nucleic acids.

Item 33. The composition of any of items 29 to 32, wherein the magnetic particles do not include a binding partner which is a nucleic acid, biotin, avidin, an antibody, an aptamer, a receptor, or a receptor ligand.

Item 34. The composition of any of items 29 to 33, wherein the chaotrope comprises one or more of: urea, guanidinium bromide (guanidine hydrobromide or guanidine monohydrobromide), guanidinium iodide (guanidine hydroiodide), guanidinium chloride (guanidine hydrochloride), guanidine thiocyanate (guanidinium thiocyanate), guanidine nitrate (guanidinium nitrate), guanidine sulfate salt (guanidinium sulfate), guanidine carbonate salt (guanidinium carbonate), sodium iodide, and sodium perchlorate.

Item 35. The composition of any of items 29 to 34, wherein the binding buffer does not include PEG.

Item 36. The composition of any of items 29 to 35, wherein the magnetic particles do not comprise carboxyl functional moieties and/or amine functional moieties.

Item 37. The composition of any of items 29 to 36, wherein the chelating agent is one or more of: diethylenetriaminepentaacetic acid (DTPA), ethylenediaminetetraacetic acid (EDTA), ethylene glycol-bis(β-aminoethyl ether)-N,N,N′,N′-tetraacetic acid (EGTA) and N,N-bis(carboxymethyl)glycine (NTA).

Item 38. The composition of any of items 29 to 37, wherein the alcohol is one or more of: methanol, ethanol and isopropanol.

Item 39. The composition of any of items 29 to 38, wherein the binding buffer comprises a buffered aqueous solution of guanidine hydrochloride and potassium acetate or sodium acetate, and has a pH in the range of about pH 4 to about pH 6.

Item 40. The composition of any of items 29 to 39, wherein the volume:volume ratio of the alcohol to the binding buffer is in the range of about 0.25:1 to about 1.75:1.

Item 41. The composition of any of items 29 to 40, wherein the volume:volume ratio of the alcohol to the binding buffer is in the range of about 1:1 to about 1.25:1.

Item 42. The composition of any of items 29 to 41, wherein the volume:volume ratio of the alcohol to the binding buffer is about 1.125:1.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of normalizing a mass amount of nucleic acids in each of a plurality of test samples, comprising:
providing a plurality of input samples containing nucleic acids in an aqueous liquid, each of the plurality of input samples present in a separate container;
adding a binding mixture to each container, producing a normalization mixture in each container, the binding mixture comprising:
a quantity of magnetic particles comprising pendant hydroxyl functional groups,
a chelating agent,
a binding buffer, and
an alcohol,
wherein the binding buffer comprises a buffered aqueous solution of a chaotrope, wherein the quantity of magnetic particles is capable of reversibly and non-specifically binding nucleic acids at a binding capacity in the range of about 1 nanogram to about 5 micrograms,
and wherein each of the plurality of input samples contains a mass amount of nucleic acids greater than the binding capacity of the quantity of magnetic particles;
incubating each normalization mixture under binding conditions, thereby reversibly and non-specifically binding a portion of the nucleic acids to the magnetic particles;
separating the magnetic particles with the reversibly and non-specifically bound nucleic acids from non-bound nucleic acids by application of a magnetic field; and
eluting the reversibly and non-specifically bound nucleic acids from the magnetic particles, producing a plurality of test samples, wherein each of the plurality of test samples comprises isolated nucleic acids having a mass amount, wherein the mass amount of the isolated nucleic acids is approximately equivalent to the binding capacity of the magnetic particles, thereby providing a normalized mass amount of nucleic acids in each of the plurality of test samples.

2. The method of claim 1, wherein no internal standard is added to the container.

3. The method of claim 1, wherein the mass amount of nucleic acids in the input samples is not quantitated.

4. The method of claim 1, further comprising pooling the plurality of test samples, producing pooled test samples, and sequencing at least some of the nucleic acids in the pooled test samples.

5. The method of claim 1, wherein the chaotrope comprises one or more of: urea, guanidinium bromide (guanidine hydrobromide or guanidine monohydrobromide), guanidinium iodide (guanidine hydroiodide), guanidinium chloride (guanidine hydrochloride), guanidine thiocyanate (guanidinium thiocyanate), guanidine nitrate (guanidinium nitrate), guanidine sulfate salt (guanidinium sulfate), guanidine carbonate salt (guanidinium carbonate), sodium iodide, and sodium perchlorate.

6. The method of claim 1, wherein the binding buffer does not include PEG.

7. The method of claim 1, wherein the magnetic particles do not comprise carboxyl functional moieties and/or amine functional moieties.

8. The method of claim 1, wherein the magnetic particles comprising pendant hydroxyl functional groups comprise a hydroxyl-functionalized spacer covalently bonded to, and extending from the magnetic particle and/or a coating of the magnetic particles, wherein the spacer comprises a chain of at least 3 atoms covalently bonded to a hydroxyl functional group.

9. The method of claim 1, wherein the chelating agent is one or more of: diethylenetriaminepentaacetic acid (DTPA), ethylenediaminetetraacetic acid (EDTA), ethylene glycol-bis(β-aminoethyl ether)-N,N,N′,N′-tetraacetic acid (EGTA) and N,N-bis(carboxymethyl)glycine (NTA).

10. The method of claim 1, wherein the alcohol is one or more of: methanol, ethanol and isopropanol.

11. The method of claim 1, wherein the binding buffer comprises a buffered aqueous solution of guanidine hydrochloride and potassium acetate or sodium acetate, and has a pH in the range of about pH 4 to about pH 6.

12. The method of claim 1, further comprising recovering the non-bound nucleic acids of one or more normalization mixtures.

13. The method of claim 1, comprising transferring the non-bound nucleic acids of one or more normalization mixtures to corresponding containers;
adding a binding mixture to each corresponding container, producing a recovery mixture in each corresponding container, the binding mixture comprising:
a quantity of magnetic particles comprising pendant hydroxyl functional groups, a chelating agent,
a binding buffer, and
an alcohol,
wherein the binding buffer comprises a buffered aqueous solution of a chaotrope,
wherein the quantity of magnetic particles is capable of reversibly and non-specifically binding nucleic acids at a binding capacity in the range of about 1 nanogram to about 5 micrograms,
and wherein each of the recovery mixtures contains a mass amount of nucleic acids greater than, less than, or equal to, the binding capacity of the quantity of magnetic particles;
incubating each recovery mixture under binding conditions, thereby reversibly and non-specifically binding all, or a portion of, the nucleic acids to the magnetic particles, producing magnetic particles reversibly and non-specifically bound to the recovered nucleic acids;
separating the magnetic particles reversibly and non-specifically bound to the recovered nucleic acids by application of a magnetic field; and
eluting the reversibly and non-specifically bound recovered nucleic acids from the magnetic particles.

14. The method of claim 1, wherein the volume:volume ratio of the alcohol to the binding buffer is in the range of about 0.25:1 to about 1.75:1.

15. A kit, comprising:
magnetic particles comprising pendant hydroxyl functional groups;
a binding buffer comprising a buffered aqueous solution of a chaotrope;
a wash liquid; and
an elution buffer.

16. The kit of claim 15, further comprising a chelating agent and an alcohol.

17. The kit of claim 15, wherein the binding buffer comprises a buffered aqueous solution of guanidine hydrochloride and potassium acetate or sodium acetate, and has a pH in the range of about pH 4 to about pH 6.

18. The kit of claim 15, wherein the elution buffer comprises 10 mM Tris-HCl, pH 8.0, and 0.1 mM EDTA.

19. The kit of claim 15, wherein the wash liquid comprises about 60% to about 100% ethanol.

20. The kit of claim 15, wherein the magnetic particles comprising pendant hydroxyl functional groups comprise a hydroxyl-functionalized spacer covalently bonded to, and extending from the magnetic particle and/or a coating of the magnetic particle, wherein the spacer comprises a chain of at least 3 atoms covalently bonded to a hydroxyl functional group.
